(19) United States
(12) Patent Application Publication
(10) Pub. No.: US 2018/0177505 A1
(43) Pub. Date: Jun. 28, 2018

(54) BARBED SUTURE

(71) Applicant: Jack CHU, Santa Rosa, CA (US)

(72) Inventor: Jack CHU, Santa Rosa, CA (US)

(21) Appl. No.: 15/393,223

(22) Filed: Dec. 28, 2016

(51) Int. Cl. A61B 17/06

(52) U.S. Cl. CPC A61B 17/06166; A61B 2017/00526; A61B 2017/06176

(57) ABSTRACT

A novel barbed suture comprises improved retaining strength or anchoring ability within the tissue in both directions of the suture. The barbed suture of the present invention comprises a plurality of barbs which are made of filaments. The ends of the filaments are attached to the main body of the suture thread to form barbs. The barbs are flexible and allow tissue ingrowth. As a result, the barbed suture is allowed to be readily deployed in the tissue with high retaining strength in both directions of the suture. The retaining strength enhances the barbed suture's ability to hold tissue together with improved wound closure or tissue approximation performance.

### CROSS REFERENCE TO RELATED APPLICATIONS

None.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present invention relates to a new barbed suture and methods to manufacture it. More particularly, the present invention relates to a new barbed suture for surgical procedures.

### BACKGROUND

Wound closure techniques have evolved from the earliest development of suturing materials to comprise resources that include synthetic sutures, resorbable sutures, staples, surgical tapes, and tissue adhesives. They have been widely used in surgical procedures such as wound closure, tissue repair, tissues approximation, anatomical space closure, or repositioning tissues to new anatomical locations, etc.

Suture is commonly used in surgery. Some of the applications don't require the use of delivery device to deliver suture in the body. However, most surgical applications generally need to use a delivery device, such as a needle, to place an attached length of suture thread in the tissue. There are many variations in suture thread such as suture materials and suture sizes for various surgical procedures. A number of different materials have been developed for suture thread, and they can be classified into two general categories, absorbable and non-absorbable sutures. Absorbable suture thread is designed to degrade and be absorbed in the body over time without the need for retrieval or secondary operation. Typical materials for absorbable suture thread comprise: Polyglycolic Acid, Polyglactin 910, Catgut, Poliglecaprone 25 and Polydioxanone. On the other hand, non-absorbable suture will remain in the body if they are not removed. Typical materials for non-absorbable sutures include: Polypropylene, Polylamide, Polyester, PVDF, silk and stainless steel. The type of suture material used depends on the particular surgical procedure, the requirements of the surgical sites and their environment, and mostly depends on the experience of the surgeons.

In a surgery using traditional suture, a needle with attached suture thread is passed through opposite edges of the wound (or tissue) to bring the wound edges together. The suture is then looped to form a knot to tie the suture thread together while holding wound edges in approximation. Tying the knot may be done inside or outside the body depending on the type of surgery. It is usually time consuming with many complications such as infection and ischemia in the tissue adjacent to the tightened knots. In addition, the suture knots create stresses and are usually the weak links in the suture which may lead to premature suture failure, wound dehiscence or tissue separation in some cases.

Due to those issues in the knot tying of traditional suture, barbed suture was developed as a knotless surgical suture. A barbed suture is generally a suture thread having a plurality of axially and circumferentially spaced barbs on the exterior surface of the suture thread. The advantage of barbed suture is that it can apply tension in the tissue with less suture slippage in the tissue. While suturing tissue, these barbs on the suture penetrate into the tissue and resist suture movement in the direction of the barb tip and lock the suture in place. As a consequence, the barbed suture enables tissue repair (or wound approximation) without the need for tying knots to fasten the suture, resulting in quicker surgical procedures. Other benefits of a barbed suture are a reduction in infection rate, less scar after healing and an improved blood supply to wound edges allowing a faster healing. In addition to wound repair and tissue approximation, barbed sutures are often used in plastic surgery for face and breast lifts. Similar to traditional suture, the barbed suture can be inserted into tissue using a typical delivery device such as needle.

Various methods have been published regarding the use of sutures with high friction. U.S. Pat. No. 3,123,077, describes a surgical suture with a roughened surface or barbs to prevent slippage of the suture after each stitch. The surgical suture is intended for use with a device such as described in U.S. Pat. No. 2,988,028, to create a running or looped stitch. However, the method for manufacturing of this surgical suture was not disclosed.

U.S. Pat. Nos. 5,342,376; 5,931,855; 6,241,747; 7,226,468; 8,246,652 and 7,806,908 describe new forms of barbed sutures which could be used for surgical procedures and plastic surgery such as facelift for removing wrinkles in a face. The barbed sutures are available in different forms and manufactured in both absorbable and non-absorbable variations. The barbed sutures comprise barbs oriented in unidirection or bi-directions along the length of the suture thread, depending on the requirements for particular surgical procedure.

The barbs on the barbed suture may be formed on a filament utilizing processes such as cutting, injection molding, extrusion, and stamping, etc. In most barbed sutures that are commercially available, the barbs are formed by cutting into the suture thread with a blade, a laser or other cutting means. For example, barbs may be formed by cutting into the suture thread in an angle, and separating the cut section from the body of the suture thread. Some typical cutting methods are disclosed in U.S. Pat. Nos. 3,720,055; 6,848,152; 7,225,512; 7,913,365; 7,996,967; 7,996,968; 8,011,072; 8,015,678; 8,020,263; 8,028,387; 8,028,388; 8,926,659; 6,848,152; 8,793,863; 8,032,996; 8,267,961; 8,715,320; 8,663,277; 8,273,105; 8,632,567; 8,739,389; 9,050,082; 8,852,232; 8,821,540; 8,875,607; 8,734,485; 8,795,332; 8,852,232; 8,747,436; 8,100,940; 9,248,580; 8,721,681; 8,641,732; and US Patent Publication Nos. 2006/0135994; 2010/0087855; 2007/0219587; 2014/0213966; and 2003/0041426. These cutting methods are currently widely used, and most of sutures for surgical procedures and plastic surgery are manufactured according to those cutting methods disclosed above.

However, there are several issues with the cutting methods to make barb. First, the barb is formed by cutting into the suture thread. As a result, high stresses are concentrated at the base of the barb. Those high stresses concentrations weaken the barb and limit the load carrying capability of the barb while the barb is engaging the tissue. Second, the diameter of the suture thread is reduced at the area where a barb has been cut. This reduced diameter greatly reduces the load bearing capability or anchoring strength of the suture thread compared with the suture without being cut. Third, the number, dimension, arrangement and geometry of the barbs determine the retaining strength of the barb suture. However, they are limited by the space available for cutting on the suture thread without adversely affects the strength of the suture. Due to those limitations described here, various other methods of forming barbs on sutures have been proposed.

U.S. Pat. Nos. 8,795,332; 8,734,485; and 8,852,232 propose manufacturing processes such as injection molding, stamping and laser cutting for a barbed suture used for surgical tissue suturing and plastic surgery. U.S. Patent Publication No. 2003/0149447, 2010/0146770, 2014/0081321 also suggest stamping, progressive die cutting, injection molding, and chemical etching as methods to produce barbed sutures out of a flat material. U.S. Pat. No. 4,069,825 suggests casting plastics to form barb suture. U.S. Pat. Nos. 7,850,894 and 8,216,497 describe barbed sutures forming methods including press forming methods, and press forming methods in combination with profile punching. U.S. Pat. No. 8,460,338 describes a method of thermal forming barbed sutures. US Patent Publication No. 2007/0239207 describes injection molded fixing claws placed alongside the thread surface. U.S. Pat. No. 5,393,475 describes a suture that is extruded from an extrusion nozzle at the predetermined width and is introduced between die rollers with a plurality of barb forming cavities. U.S. Pat. No. 9,186,135 describes a barb suture manufacturing steps of thermal forming, twisting under a tensile and a rotational force to the suture in a vacuum.

Each of these suggested methods described above has its drawbacks. The major factor for surgical suture's high tensile strength is the molecular orientation formed in the resin during the extrusion, drawing and cooling processes. Even though injection molding process can melt the resin to high temperature allowing it to flow into a die and forming the desire shape, it can't achieve the degree of molecular orientation and strength obtained in typical suture manufacturing processes. Thermal forming process also involves melting or heating the resin to high temperature allowing it to flow into a die to form the desire barbs. Significant heat exposure in this process decreases the amount of molecular orientation in the resin as the polymer chains are able to change into a more random molecular structure. The loss of molecular orientation can adversely affect suture's mechanical properties such as tensile strength and modulus, which can also reduce the retaining strength of the suture in the tissue.

In an effort to avoid significant amount of thermal treatment or heat exposure involved in the barb forming process on the suture, U.S. Pat. No. 9,011,133 describes a method to form retainers by punching suture body. U.S. Pat. No. 8,216,497 describes a method to form retainers by press forming, and press forming in combination with profile punching. U.S. Pat. No. 8,771,313 describes a method to form retainers by temporarily contacting the suture body with heated element. US Patent Publication No. 2013/0226234 describes a self-retaining braided suture having a braided core and an outer sheath in which the retainers are formed from the material of the outer sheath. U.S. Pat. No. 8,663,277 describes a suture assembly including a plurality of unbarbed filamentary elements intertwined with one another and at least one barbed filamentary element having plurality of barbs extending outwardly. U.S. Pat. No. 8,414,612 describes a surgical suture including a plurality of intertwined unbarbed filaments and a barbed filament disposed along a length and partially intertwines with the unbarbed filaments. US Patent Publication No. 2010/0298872 describes a surgical suture including filamentary elements projecting from the filamentary braid for anchoring in biological tissue. U.S. Pat. No. 4,662,886 describes a suture comprising a core of a multiplicity of flexible filaments, and partial sheathing interwoven in contra-rotation so that areas of the core are exposed at frequent intervals, to encourage penetration and ingrowth of fresh tissue between the core filaments. However, each of these suggested methods described above has its drawbacks. In some cases, the diameter of the suture thread is reduced at the area where a barb has been formed. Or some of the filaments are projecting from multi-filament suture and used to increase friction on the suture surface. Both processes reduce the load bearing capability or anchoring strength of the suture thread. In addition, the friction enhanced features described in those disclosures are usually insufficient or less effective in gaining retaining strength for the suture.

Some other patent publications disclose methods to attach features on the suture thread to increase friction without changing the suture thread much. U.S. Pat. No. 8,916,077 describes a method to form barbs by applying molten material to the periphery of the suture body and cooling the material to form the retainers. U.S. Pat. Nos. 9,241,709; 9,168,036; 8,640,331 describe a barb with legs which can be used to attach barb to a suture. U.S. Pat. Nos. 5,123,913; 4,950,285; 4,069,825; 7,468,068; 7,582,105; 8,226,684 disclose suture assemblies including a plurality of frusto-conically shaped tissue-engaging elements in a generally spaced arrangement alone the suture. U.S. Pat. No. 8,876,865 discloses sutures including bi-directional retainers, each of which can be deployed in two directions, but once deployed in one direction, resist motion in the opposite direction. Other sutures include uni-directional retainers that are conical in shape, and include tissue engaging protrusions that extend from edges of the conical retainers. U.S. Pat. No. 9,011,489 and EP Patent No. 1,075,843 disclose a filament including a retaining member extending from the filament. The retaining member is coupled to the filament and configured to anchor the filament. International Patent Publication No. WO2011/034286 describes surgical suture thread comprising a core filament and an anti-slip outer filament wound into a spiral in the lengthwise direction of the core and fixed at the core. U.S. Pat. No. 9,044,224 describes barbed medical devices including a multifilament elongate body and a monofilament fragment. The monofilament fragment positioned within the multifilament elongate body and extends beyond the outer surface of the multifilament elongate body to form a barb. International Patent Application No. WO2015/046865 discloses a barbed suturing thread having a plurality of engraved helical belt formed on the outer circumferential surface of the suture. U.S. Patent Publication No. 2015/0073474 discloses a barbed suture including a core fiber and a surrounding sheet with a plurality of barbs extending outwardly from the sheet. However, some of those barbs disclosed in those methods are too bulky and may cause damage to the tissue during the suture placement procedures. Other barbs designs are too expensive to manufacture or are relatively weak with insufficient anchoring capability. As a result, there is a need to improve the performance of barbed suture so that it can overcome those deficiencies disclosed in the prior arts and to provide a barbed suture with sufficient retaining strength and manufacturability without the need for tying knots.

### SUMMARY OF THE DISCLOSURE

This invention is about a new barbed suture and methods to manufacture it. In the present invention, the new barbed suture comprises a suture thread and a plurality of barbs attached to the suture thread. Each barb comprises a substantially V-shaped filament having an angle at the peak and two ends. Both ends of each V-shaped filament are attached to the main body of the suture thread and spaced apart from each other. The peak of the V-shaped filament forms the barb tip which is configured to engage tissue and anchor the barbed suture in the tissue. In addition, the barb tip points away from the insertion end of the suture thread in an acute angle with the central axis of the suture thread.

The barb of the present invention is resilient, flexible and operative to collapse and conform to the suture thread upon contacting with the tissue while the barbed suture is inserted through the tissue. As a consequence, the barbed suture is deliverable in the tissue. Following its passage through the tissue, the barb is also operative to expand and resist suture movement in the same direction as the barb tip.

In addition, the barb of the present invention allows tissue to grow into and around the barb with enhanced tissue fixation. This enhanced tissue fixation is against suture movement in either direction of the suture thread. Therefore, this innovative barbed suture enables suture retention in both directions of the suture thread with improved wound closure or tissue approximation performance.

In another embodiment of the present invention, the barb formed by the filament comprises excellent resistance against moving in the same direction as the barb tip. Because both ends of the barb are attached to the suture thread, a high bending force is required to deform and dislodge the barb from the tissue. As a consequence, the barb suture disclosed in this invention comprises higher retaining strength in the tissue compared with barb suture having only single barb attachment point on the suture thread.

In another embodiment of the present invention, the new barbed suture comprises barbs which are attached to the suture thread without cutting into the suture thread or reducing the diameter and strength of the suture thread.

In further embodiment of the present invention, the barb of the new barbed suture comprises a plurality of inter-connected barbs with enhanced barb strength.

According to further embodiment of the present invention, the disposition of the successive barbs on the suture thread can be wound spirally around the suture thread with space between each turn. Or the successive barbs can also be placed circumferentially around the suture surface with space between each turn. Alternatively the successive barbs can be axially spaced apart along the central axis of the suture thread. Further, the successive barbs can be circumferentially disposed around the suture thread and axially spaced apart along the central axis of the suture thread.

In another embodiment of the present invention, the suture thread of the new barbed suture is either monofilament or multi-filaments. In yet another embodiment of the present invention, the multi-filaments suture thread of the new barbed suture is either braided or non-braided.

In further other embodiment of the present invention, the barbs on the suture thread are either unidirectional or bidirectional.

In yet another embodiment of the present invention, the barbed suture further comprises an insertion device, such as a needle, that is linked to one end of the barbed suture. In another embodiment of the present invention, the barbed suture further comprises two insertion devices that are linked to both ends of the barbed suture.

According to another embodiment of the present invention, the present invention provides methods to manufacture the barbed suture. One exemplary method is: First, the suture thread and the filament are extruded from an extruder. Then the filament is bent by a fixture to form a V shape. Two ends of the V shaped filament are attached to the main body of the suture thread by typical bonding process to form a barb in an acute angle to the suture thread.

According to other embodiments of the present invention, the barb suture of the present invention comprises a medicament to promote healing, treat diseases, prevent infection, reduce pain, reduce scar, etc.

In the other embodiment of the present invention, the new barbed suture further comprises a binder which is water soluble or degradable. The binder is able to temporarily bond the barbs to the main body of the suture thread. By binding and restraining those barbs to the main suture thread, the barbs wouldn't penetrate the tissue, extend radially outwards or “stand up” to raise the cross-sectional profile of the barbed suture, thereby increasing resistance against needle pulling during the insertion of the barbed suture in the tissue. After the suture is placed in the tissue, this binder will be dissolved by body fluid or degraded in the body releasing the barbs on the suture. Those released barbs can resist suture movement in the same direction as the barb tips. Therefore, the new barbed suture can be easily delivered into the tissue to hold tissue together with improved wound closure or tissue approximation performance.

In one embodiment of this invention, this biocompatible binder is soluble or degradable in water or body fluid. The binder is able to temporarily bind the barb with the main body of the suture thread. The examples of water soluble binder include saccharides and their derivatives, protein, surfactants, adhesives, gums, water soluble polymers, Tragacanth, minerals, etc.

According to one embodiment of the present invention, the present invention provides methods to apply the binder to the barbed suture. The binder can be dissolved in a solvent to form a solution. Then this solution is applied on the suture by coating, spraying, brushing or dipping to cover the barbs or bond the barbs to the main suture thread.

According to the other embodiment of the present invention, the present invention provides methods to apply the binder to the selected barbs on the barbed suture. A first set of barbs on the suture can be formed either by cutting into the suture thread or by attaching features on the suture thread. Then the binder can be applied on the suture by spraying, brushing or dipping to bind the first set of barbs to the main suture thread. After the binding is completed, a second set of barbs are formed on different sites of the same barbed suture. The second set of barbs can be formed either by cutting into the suture thread or by attaching features on the suture thread.

According to yet the other embodiment of the present invention, the present invention provides methods to apply the binder to the selected barbs on the barbed suture. After the barbs are formed on the suture thread, some of the barbs are covered by a protective sheet or a protective coating to mask and prevent those barbs from contacting with the binder. As a consequence, only the selected barbs are exposed to the binding processes. Then, the binder is applied on the suture by spraying, brushing or dipping to bind the exposed barbs to the main suture thread.

In summary, according to the embodiments of the present invention, the present invention provides new methods to make new barbed sutures with enhanced flexibility, improved retaining strength or anchoring ability within the tissue in both directions of the barbed suture.

In some embodiments, a barbed suture is provided. The barbed suture includes a suture thread with a central axis, and a plurality of barbs disposed on a surface of the suture thread. In some embodiments, each barb includes a substantially V-shaped filament with a peak and two ends. The two ends of the filament can be attached to the suture thread and spaced apart from each other. The peak of the filament is configured to penetrate tissue. Each barb can be oriented in an acute angle to the central axis of the suture thread.

In some embodiments, the successive barbs of the plurality of barbs are circumferentially disposed around the suture thread.

In some embodiments, the successive barbs of the plurality of barbs are axially spaced apart along the central axis of the suture thread.

In some embodiments, the successive barbs of the plurality of barbs are circumferentially disposed around the suture thread and axially spaced apart along the central axis of the suture thread.

In some embodiments, the successive barbs of the plurality of barbs are disposed spirally on the surface of the suture thread.

In some embodiments, the ends of each barb are connected to the ends of adjacent barbs.

In some embodiments, at least one barb overlaps with an adjacent barb on the surface of the suture thread.

In some embodiments, the plurality of barbs are interconnected.

In some embodiments, the plurality of barbs overlap and form a lattice structure.

In some embodiments, each barb forms an opening that is configured to allow tissue ingrowth.

In some embodiments, the two ends of the filament are attached to the suture thread by a method selected from contact welding, ultrasonic welding, hot air welding, induction welding, speed tip welding, RF welding, friction welding, laser welding, and adhesive.

In some embodiments, successive barbs of the plurality of barbs are separated by a predetermined distance along a length of the suture thread, the predetermined distance between approximately 0.01 to 1.0 cm.

In some embodiments, the length of each barb is between approximately 0.01 to 1.0 cm.

In some embodiments, the thickness of each barb is between approximately 0.01 to 0.8 cm.

In some embodiments, the ends of the V-shaped filament are spaced apart at a distance approximately ¼, ⅓, ½, ⅔ or 3/3 of the width of the suture thread.

In some embodiments, the suture thread and/or the plurality of barbs includes material selected from the group consisting of degradable polymer, non-degradable polymer, metal, metal alloy, ceramic, silk, and mixtures thereof.

In some embodiments, the degradable polymer is selected from the group consisting of polylactic acid polymer or copolymer, polyglycolic acid polymer or copolymer, copolymers of polylactic acid and polyglycolic acid, polyepsilon caprolactone polymer or copolymer, poly caprolactone polymer or copolymer, polyhydroxy butyric acid polymer or copolymer, poly-3-hydroxybutyrate polymer or copolymer, polyorthoesters polymer or copolymer, polyacetals polymer or copolymer, polydihydropyrans polymer or copolymer, collagen, hydrogel, hyaluronic acid, polyacrylamine, poly(vinylcarboxylic acid), polymethacrylic acid, polyacrylic acid polymer or copolymer, poly amino acids, hydrogel, fibrin, chitosan, gelatin, alginate, cellulose acetate phthalate, cellulosic, polylysine, polyarginine, poly aspartic acid, poly glutamic acid, methylcellulose, hydroxy ethyl cellulose, hydroxy propyl cellulose, hydroxypropyl methylcellulose, cellulose acetate, nitrocellulose, hypromellose, cellulose butyrate, polyhydroxyalkanoates, polyhydroxyvalerate (PHV) and polyhydroxyhexanoate, polyanhydrides, polyethylene terephthalate, polyhydroxurethanes, poly(2-hydroxyethyl-methacrylate), polyphosphazenes, polyphosphoesters, polyglactin, catgut, silk, poliglecaprone, polydioxanone, poly urethane, ethylene vinyl alcohol copolymers, polyacrylonitrile, polyvinylacetate, copolymers of urethane/carbonate, copolymers of styrene/maleic acid, and mixtures or copolymer thereof.

In some embodiments, the non-degradable polymer is selected from the group consisting of polypropylene, polyethylene, polyamide, polyester, polytetrafluoroethylene, polyether-ester, or polyurethane, and mixtures thereof.

In some embodiments, the barbed suture further includes a binder. The binder can temporarily bind the plurality of barbs to the suture thread into a delivery configuration where the plurality of barbs are pressed against the surface of the suture thread. The binder can be soluble or degradable in a patient's body and configured to release the plurality of barbs into a deployed configuration where the plurality of barbs are configured to extend radially outwards in order to resist movement of the suture thread in the patient's body.

In some embodiments, the binder is selected from the group consisting of saccharides and their derivatives, proteins, surfactants, adhesives, gums, water soluble polymers, minerals, etc. and mixtures thereof.

In some embodiments, the saccharides is selected from the group consisting of monosaccharides, glucose, fructose, galactose, disaccharides, sucrose, maltose, lactose, polysaccharides, starches, modified starch, pregelatinized starch, cellulose or modified cellulose, methyl cellulose, ethyl cellulose, ethyl methyl cellulose, hydroxyethyl cellulose, hydroxyethyl methylcellulose, hydroxyl propyl methylcellulose, ethyl hydroxyl ethyl cellulose, microcrystalline cellulose, sodium carboxymethyl cellulose, hydroxypropyl cellulose, carboxymethyl cellulose, cellulose ethers, Hypromellose, sugar alcohols, xylitol, sorbitol or maltitol, glycerol, erythritol, threitol, arabitol, ribitol, mannitol, galacitol, fucitol, and mixtures thereof.

In some embodiments, the gum is selected from the group consisting of agar, alginic acid, Tragacanth, sodium alginate, carrageenan, acacia, gum Arabic, gum copal, kauri gum, spruce gum, gum ghatti, gum tragacanth, karaya gum, guar gum, locust bean gum, chicle gum, dammar gum, mastic gum, Xanthan gum, etc. and mixtures thereof.

In some embodiments, the water soluble polymers is selected from the group consisting of partially deacetylated polyvinyl alcohol, ethylene-vinyl alcohol copolymers, poly-3-hydroxybutyrate, cellulose acetates, hydrogels, polyvinyl pyrrolidone (PVP) polymer or copolymer, Copolyvidone, polyethylene oxide (PEO) polymer or copolymer, poly(ethylene glycol), poly(propylene glycol), polytetramethylene oxide, poly vinyl alcohol (PVA) polymer or copolymer, polymethacrylate, Carbopol, Poloxamer, Tetronics, polyhema polymer or copolymer, Hypan polymer or copolymer, poly(hydroxy ethyl acrylate), poly(hydroxy ethyl methacrylate), hydroxy ethyl cellulose, hydroxy propyl cellulose, hydroxypropyl methylcellulose, hypromellose, methylcellulose, collagen, hyaluronic acid, poly amino acids, fibrin, gelatin, alginate and mixtures thereof.

In some embodiments, the barbed suture includes materials selected from absorbable materials or non-absorbable materials.

In some embodiments, the barbed suture is a unidirectional suture. In some embodiments, the barbed suture is a bidirectional suture.

In some embodiments, the barbed suture further includes at least one delivery device. In some embodiments, the delivery device is a needle.

In some embodiments, the suture thread is a monofilament. In some embodiments, the suture thread is made of multifilaments.

In some embodiments, the ends are spaced apart at a distance approximately the width of the suture.

In some embodiments, the barbed suture further includes a contrast agent.

In some embodiments, the barbed suture further includes a medicament. In some embodiments, the medicament is selected from the group consisting of angiogenesis inhibiting compounds, steroidal or non-steroidal anti-inflammatory agents, thrombotic agents, growth factors, anti-proliferative agents, anti-infective agents, fibrosis-inducing agents, anti-scarring agents, analgesics, and anti-microtubule agents, etc. and mixtures thereof.

In some embodiments, a barbed suture is provided. The barbed suture includes a suture thread having a central axis, and a filament having a plurality of peaks and valleys. The filament can be disposed on a surface of the suture thread. The valleys of said filament are attached to the suture thread such that the peaks of the filament form barbs that are oriented in an acute angle to the central axis of the suture thread. The barbs are configured to penetrate tissue.

In some embodiments, the barbed suture further includes a binder, where the binder temporarily binds the filament to the suture thread into a delivery configuration where the barbs of the filament are pressed against the surface of the suture thread. The binder is soluble or degradable in a patient's body and configured to release the barbs into a deployed configuration where the barbs are configured to extend radially outwards in order to resist movement of the suture thread in the patient's body.

In some embodiments, a barbed suture is provided. The barbed suture includes a suture thread with a central axis and a surface, and a plurality of barbs disposed on the surface of the suture thread. Each barb includes a filament having a tissue penetration feature and two ends. The two ends of the filament are attached to the suture thread and spaced apart from each other. Each barb can be oriented in an acute angle to the central axis of the suture thread such that the barb is configured to collapse upon contact with the tissue during insertion. The filament is configured to allow tissue to grow into space between the filament and the suture thread.

In some embodiments, the barbed suture further includes a binder, where the binder temporarily binds the barbs to the suture thread such that the suture is configured to be inserted into tissue. The binder is soluble or degradable in the body to release the barbs, and the released barbs resist movement of the suture in the body.

In some embodiments, the barbed suture further includes connections at the ends of successive barbs.

In some embodiments, a method to make a barbed suture is provided. The method includes forming a suture thread having a central axis and a surface; forming a substantially V-shaped filament having a tissue penetration peak and two ends; and attaching the two ends of the filament to the surface of the suture thread such that the two ends are spaced apart from each other by a predetermined distance and the peak is oriented at an acute angle to the central axis of the suture thread.

In some embodiments, the method further includes applying a binder to temporarily bind the filament to the suture thread.

In some embodiments, a method to make a barbed suture is provided. The method includes forming a suture thread having a central axis and a surface; forming a zigzag filament having a plurality of peaks and valleys along the length of the filament, where the peaks are configured to penetrate tissue; attaching the valleys of the filament to the surface of the suture thread; and orienting the peaks of the filament in an acute angle to the central axis of the suture thread.

In some embodiments, the method further includes applying a binder to temporarily bind the filament to the suture thread.

### BRIEF DESCRIPTION OF THE DRAWINGS

FIG. 1 illustrates a prospective view of an unidirectional barbed suture having a plurality of barbs attached to the suture thread.

FIG. 2 illustrates a V-shaped barb used on various embodiments of the barbed suture disclosed in this invention.

FIG. 3 is a cross-sectional view of the unidirectional barbed suture disclosed in this invention from line AA in FIG. 1.

FIG. 4 illustrates a prospective view of another unidirectional barbed suture having a plurality of barbs attached to the suture thread.

FIG. 5 is a cross-sectional view of the unidirectional barbed suture from Line BB disclosed in FIG. 4.

FIG. 6 illustrates a prospective view of yet another unidirectional barbed suture disclosed in this invention.

FIG. 7 is a cross-sectional view of the unidirectional barbed suture from Line CC disclosed in FIG. 4.

FIG. 8 illustrates a prospective view of further another unidirectional barbed suture disclosed in this invention.

FIG. 9 is a cross-sectional view of the unidirectional barbed suture from Line DD in FIG. 8 after the barbs are attached to the suture.

FIG. 10 illustrates a zigzag filament which can be used to make barbs on various embodiments of the barbed suture disclosed in this invention.

FIG. 11 illustrates an exemplary manufacture process that a zigzag filament (illustrated in FIG. 10) is wound around the suture thread to make an unidirectional barbed suture disclosed in this invention.

FIG. 12 illustrates a prospective view of another unidirectional barbed suture disclosed in this invention.

FIG. 13 is a cross-sectional view of the unidirectional barbed suture from Line EE in FIG. 12 after the barbs are attached to the suture.

FIG. 14 illustrates a prospective view of yet another unidirectional barbed suture disclosed in this invention.

FIG. 15 is a cross-sectional view of the unidirectional barbed suture from Line GG in FIG. 14 after the barbs are attached to the suture.

FIG. 16 illustrates another zigzag filament which can be used to make barbs on a barbed suture disclosed in this invention.

FIG. 17 illustrates yet another zigzag filament which can be used to make barbs on a barbed suture disclosed in this invention.

FIG. 18 illustrates a barb which can be used on a barbed suture disclosed in this invention.

FIG. 19 illustrates a continuous barbs string which can be used on a barbed suture disclosed in this invention.

FIG. 20 illustrates a continuous barbs lattice which can be used on a barbed suture disclosed in this invention.

FIG. 21 illustrates a prospective view of yet another unidirectional barbed suture disclosed in this invention.

FIG. 22 is a cross-sectional view of the unidirectional barbed suture from Line HH in FIG. 21 after the barbs are attached to the suture.

FIG. 23 illustrates a continuous barbs structure which can be used on a barbed suture disclosed in this invention.

FIG. 24 illustrates a prospective view of a bidirectional barbed suture disclosed in this invention.

FIG. 25 illustrates a prospective view of an unidirectional barbed suture disclosed in this invention.

FIG. 26 illustrates a prospective view of a bidirectional barbed suture disclosed in this invention.

FIG. 27 illustrates a prospective view of the bidirectional barbed suture disclosed in FIG. 26 after some of its barbs are bound to the suture thread.

FIG. 28 illustrates a prospective view of the bidirectional barbed suture disclosed in FIG. 26 after its barbs are bound to the suture thread.

FIG. 29 illustrates a prospective view of a bidirectional barbed suture disclosed in this invention.

FIG. 30 illustrates a prospective view of the bidirectional barbed suture disclosed in FIG. 29 after some of its barbs are bound to the suture thread.

### DETAILED DESCRIPTION

### Definitions

Definitions of the terms that may be used in this invention include the following:

“Barbed suture system” refers to a barbed suture combined with a delivery device or devices for placing the barbed suture into the tissue. Such delivery devices may include, without limitation, suture needles with sufficient rigidity and sharpness to assist the penetration of barbed suture in the tissue. Each barbed suture system may comprise one or more delivery devices. Certain delivery devices may also prevent barbs from contacting with tissue during the insertion process of the suture into the tissue.

“Barbed suture” refers to a suture that comprises suture thread and features or attachments on the main suture thread to engage the tissue anchoring the suture in the tissue. The suture thread has at least two ends with at least one central axis.

“Barb” refers to a feature of a suture thread which is configured to engage tissue and resist movement of the suture thread in at least one axial direction. For example, barbs can include hooks, projections, bulges, anchors, surface roughness, surface irregularities, surface defects, etc. Because the need to insert the suture in the tissue, the barbs are pointed substantially away from the insertion direction to avoid engaging the tissue during the insertion and resisting moving. In some embodiments, the barbs conform to the main body of the suture thread when pulled in the insertion direction. Once the barbed suture has been deployed in the tissue, a pulling force exerted opposite to the insertion direction will cause the barbs (and barb tips) to move from the deployment position to penetrate into the surrounding tissue. The penetration of barb tips into the surrounding tissue results in tissue being caught between the barbs and the suture thread. Further movement in that direction will force the barb tip to “stand up” or extend radially outwards and penetrate even deeper into the surrounding tissue. This will increase the cross sectional profile of the suture and its resistance against movement opposite to the insertion direction, therefore anchoring the barbed suture in the same position in the tissue. In some other embodiments, the barbs on the barbed suture are configured to allow suture movement in one direction and resist suture movement in the opposite direction. In certain other embodiments, the barbs on the barbed suture are configured to resist suture movement in both directions of the suture.

“Unidirectional suture” refers to a barbed suture comprising barb tips pointed in one direction of the suture thread. A unidirectional suture is typically jointed with a needle at insertion end of the suture thread. The suture comprises barbs pointed away from the needle to reduce its resistance against insertion. As a consequence, the unidirectional barbed suture is allowed to move through tissue during insertion. When the suture is placed in the tissue, the barbs can resist movement of the suture in a direction opposite to the insertion direction which is also the same direction as the barb tips. When pulled in this direction, the barb tips either penetrate into the tissue anchoring the suture or “standing up” or extend radially outwards from the suture thread with a larger cross sectional area resisting against moving. However, because barbs are pointed away from the insertion direction, the suture has low resistance against moving in the insertion direction in the tissue. Even though it is beneficial to have low resistance in the insertion direction during the insertion of the suture in the tissue, a high resistance against movement in the tissue is desired in both directions of the suture. As a consequence, in certain embodiments, a stop or a loop is built at the end of the suture thread (usually the opposite end from the insertion end) to resist moving in the insertion direction.

“Bidirectional suture” refers to a barbed suture comprising barbs with barb tips pointed in both directions of the suture. In certain bidirectional sutures, they have barb tips pointed to one direction in a section of the suture, and barb tips pointed to the other direction in another section of the suture. For traditional bidirectional barbed suture, it comprises two suture sections with barbs pointed to opposite direction in these two sections. At the place two sections join together, i.e. the transition segment, the barb tips reverse direction. Each suture section is jointed with a needle at the end of the suture thread that comprises barbs pointed away from the needle. During the insertion of the suture in the tissue, because the barb tips are pointed away from the insertion direction without penetrating the tissue, the suture has low resistance against moving until the transition segment is reached. As a consequence, this section of barbed suture up to the transition segment is allowed to move through tissue during insertion. Then the other needle on the other end of the suture can do the same thing to penetrate the tissue until the transition segment is reached. While the suture is already placed in the tissue, the barbs can resist movement of the suture in the same direction as the barb tips. When the suture is pulled in the tips direction, the barb tips either penetrate into the tissue anchoring the suture or “stand up” or extend radially outwards from the suture thread with a larger cross sectional area resisting against moving. As a result, this bidirectional suture provides resistance in both direction of the suture thread, and this two needles delivery system allows the insertion of this bidirectional barbed suture in the tissue. However, the two needles delivery system requires more complex procedures to operate compared with one needle delivery system.

“Suture thread” refers to the main load carrying body of the suture. The suture thread may be made of monofilament or multiple filaments as in a braided suture. The suture thread may be made by any suitable biocompatible material. Its major function is to provide strength to approximate wound, reposition tissue or attach object to the tissue.

“Monofilament suture” refers to a suture comprising a single filamentary suture thread.

“Multiple filaments suture” refers to a suture thread comprising more than one filament. The filaments in such suture threads are typically braided, twisted, or woven together.

“Absorbable suture” refers to a suture which is made by absorbable material and is able to degrade and absorbed by the body after it is introduced in a body. Absorbable sutures lose strength and its ability to approximate tissue as the material degrades. It can be in either a braided multifilament form or a monofilament form. Absorbable suture material may include polymers such as polyglycolic acid, polylactic acid, Monocryl, polydioxanone, copolymers of glycolide and lactide, copolymers of trimethylene carbonate and glycolide with diethylene glycol, terpolymer composed of glycolide, trimethylene carbonate, and dioxanone, copolymers of glycolide, caprolactone, trimethylene carbonate, and lactide.

“Degradation” refers to a chain scission process by which a large molecular chain is cleaved into smaller molecular such as oligomers and monomers. Chain scission may occur by various mechanisms such as hydrolysis, oxidation, thermal, enzymatic or photolytic process.

“Non-absorbable suture” refers to a suture comprising material that is not degraded by chain scission such as hydrolysis, oxidation, thermal, enzymatic or photolytic process. Non-absorbable suture material includes polyamide, polyester, polytetrafluoroethylene, polyether-ester such as polybutester (block copolymer of butylene terephthalate and polytetra methylene ether glycol), polyurethane, polypropylene, polyethylene, metal alloys, metals, silk, and cotton. Sutures made of non-absorbable suture material are suitable for applications in which the suture is meant to remain permanently or is meant to be physically removed from the body later after the surgery.

“Suture insertion end” refers to an end of the suture to be inserted into the tissue. One or both ends of the suture may be suture insertion ends. The suture insertion end may be attached to a delivery device such as a needle, or the suture insertion end may be sharp and rigid enough to penetrate tissue without a delivery device.

“Staggered” and “Staggering” refers to the disposition configuration of barbs tips on a suture thread. At least two sets of barbs tips are on the suture, and they are offset with respect to each other, where the first set is aligned longitudinally at the first location on the suture and the second set is aligned longitudinally at the second location on the suture. From a cross sectional view of the barbed suture, the first location on the suture doesn't intersect the second location on the suture.

“Overlap” refers to the disposition configuration of barbs on a suture thread. At least two sets of barbs are disposed on the suture, and one is at least partially covered by the other.

This invention is about a new barbed suture and its manufacturing method. Even though there are many advantages of barbed sutures, there remains a need to improve the current barbed suture on the market so that the limitations on the current barbed suture design can be resolved. Specifically, those limitations can be addressed by the embodiments of this invention, including, but not limited to: (i) inadequate retaining strength in both directions along the barbed suture; (ii) inadequate retaining strength provided by the barb with single attachment point on the barbed suture; (iii) high stresses concentrations and weak points created by cutting barbs into the suture thread reduce the load holding and wound closure capability of the suture thread; (iv) the retaining strength of the barb suture is determined by the number, size, location and geometry of the barbs which are, in turn, limited by the space available for cutting on the suture thread without adversely affects the strength of the suture.

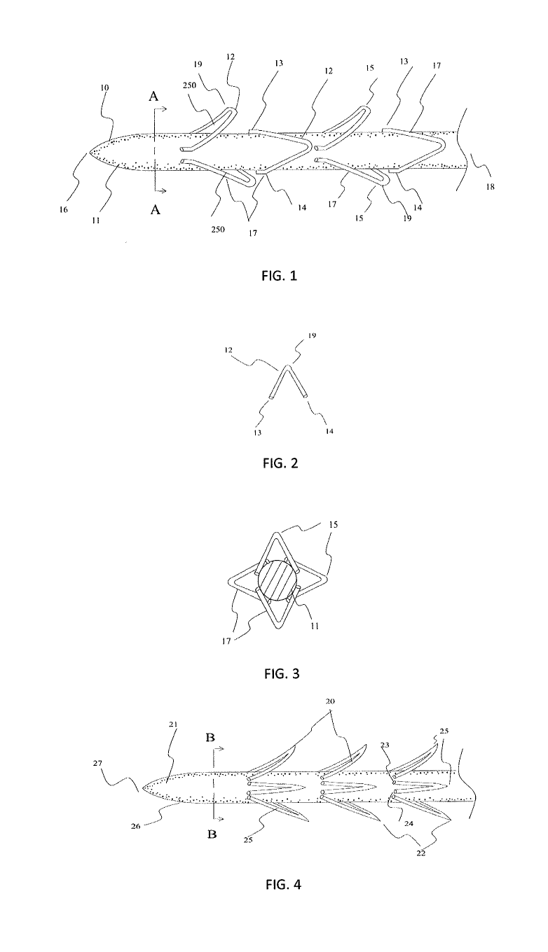

In the present invention, the new barbed suture 10 comprises a suture thread 11 with a central axis, two ends 16, 18 and a plurality of barbs 17 attached to the suture thread 11 as shown in FIG. 1. Each barb 17 comprises a substantially V-shaped filament 12 having an angle at the peak 19 and two ends 13, 14 as shown in FIG. 2. Both ends 13, 14 of each V-shaped filament 12 are attached to the main body of the suture thread 11 and spaced apart from each other as illustrated in FIG. 1. The peak 19 of the V-shaped filament 12 forms the barb tip 15 which is configured to engage tissue (not shown) and anchor the barbed suture 10 in the tissue. The angle at the peak 19 of V-shaped filament 12 can be either an obtuse or an acute angle. In addition, the barb tip 15 points away from the insertion end 16 of the suture thread 11 in an acute angle with the central axis of the suture thread 11. Because the barb 17 is made of filament 12 with space between two ends 13, 14, it is resilient and flexible. The barb 17 is operative to collapse and conform to the suture thread 11 upon contacting with the tissue while the barbed suture 10 is inserted through the tissue. As a consequence, the barb 17 doesn't penetrate the tissue or extend radially outwards against suture 10 movement, and the resulting barbed suture 10 comprises excellent deliverability in the tissue. FIG. 3 is the cross sectional view of the barbed suture 10 from Line A-A. The barbs tips 15 are staggered at approximately 90 degree intervals.

In another embodiment of the present invention, the barb 17 formed by the filament 12 comprises excellent resistance against moving in the direction of the barb tip 15 after the barbed suture 10 is inserted in the tissue (not shown). While the barbed suture 10 is pulled in the direction of the barb tip 15 in the tissue, barb tip 15 penetrates the tissue and extends radially outwards or “stands up” to raise the cross-sectional profile of the barbed suture 10, thereby increasing the resistance against pulling in the direction of the barb tip. However, if the pulling force exceeds the retaining strength of the barbed suture, the barbs on the suture will bend, and finally dislodge from the tissue. In this invention, because both ends 13, 14 of the barb 17 are attached to the suture thread 11, the barb 17 comprises higher bending strength and resistance against moving compared to a barb with only a single attachment to the suture thread that is found in traditional barbed sutures. As a consequence, the barbed suture 10 of this invention comprises higher suture retention strength in the tissue than traditional suture. A higher pulling force on the suture 10 in the barb tip 15 direction is required to deform and dislodge the barb 17 from the tissue.

Even though the suture thread 11 described here is in a preferred embodiment comprising a circular cross section, the suture thread 11 could also have a non-circular cross sectional shape that could facilitate the attachment of the barbs 17 on the surface of the suture thread 10. Other cross sectional shapes may include, but are not limited to, oval, triangle, rectangular, square, parallelepiped, trapezoid, rhomboid, pentagon, hexagon, cruciform, etc. Typically, suture threads are formed by extrusion process using a die with a circular cross section. Therefore, the cross section of the extruded suture thread becomes circular. However, any desired cross-sectional shape can be extruded with the appropriate die. These additional shapes may provide a flatter surface that facilitates barb attachment.

In yet another embodiment of the present invention, the barb 17 described here is in a preferred embodiment comprising a circular cross section. However, the barb 17 could also have a non-circular cross sectional shape that could facilitate the penetration of the barbs 17 in the tissue. Other cross sectional shapes may include, but are not limited to, oval, rectangular, triangle, square, parallelepiped, trapezoid, rhomboid, pentagon, hexagon, cruciform, etc. For example, in some embodiments, the cross sectional shape can be selected to reduce the cross-sectional area, thereby improving penetration, while increasing the surface area of the barb, thereby increasing pulling resistance and increasing the suture retention strength facilitating barb attachment to the suture. For example, the barbs can be cut from a thin sheet of material into v-shaped filaments. The surface area of the barbs can be controlled by increasing or decreasing the width of the v-shaped filaments, and the cross-sectional area is determined by the thickness and width of the filaments.

In another embodiment of the present invention, the barbed suture 10 comprises a suture thread 11 and a plurality of barbs 17 projecting from the suture thread 11. Each barb 17 is attached to the suture thread 11 and configured to resist suture movement in the barb tip 15 direction without the need to cut into the suture thread 11. Therefore, there is neither suture 11 diameter reduction, nor high stress concentration and weak point in the barbed suture 10. Furthermore, because the number, size, location and geometry of the barb 17 is no longer determined by the cutting space available on the suture thread 11, the barbed suture 10 in this invention has the advantage to enhance the performance of barbed suture 10 by optimizing the number of the barb 17 on the suture thread 11, and their configuration such as barb length, barb thickness, barb width, barb angle with the axis of suture thread 11, barb tip angle, bond length with suture thread 11, connection with adjacent barb 17, etc. By altering the number of the barbs 17, their configuration and disposition on the suture 10, the barb suture 10 can be designed for various surgical applications. For example: barb suture 10 with larger barbs 17 is desired for approximating fat and soft tissue. On the contrary, smaller barbs 17 are more suited for collagen rich tissues. In another embodiment of the present invention, the barbed suture 10 comprises barbs 17 with various configurations along the length and circumference of the suture thread.

As shown in FIG. 1, the barbs 17 can project from the exterior surface of the suture thread 11 on which the barbs 17 are disposed. In one embodiment, the length of the barbs 17 may be within the range of approximately 0.01 to 1.0 cm, and may further be approximately 0.2 cm. In yet another embodiment, the thickness of each barb 17 is within the range of approximately 0.01 to 0.8 cm, and may further be approximately 0.05 cm.

In yet another embodiment of the invention, the barbs tip 15 may be constructed in many different configurations. In an exemplary embodiment shown in FIG. 2, each barb 17 is substantially V-shaped (i.e. triangular or wedge-shaped) having an angle at the peak 19 and two ends 13, 14. The angle at the barbs tips 15 of each barb 17 is approximately within the range of approximately 0-170 degrees, and may further be approximately 25 degrees.

In another embodiment of the invention, the barb tip 15 of each barb 17 diverges from the central axis of the suture thread 11 at an acute angle within the range of approximately 0-90 degrees, and may further preferably be approximately 25-30 degrees relative to the central axis of the suture thread 11.

It would be desirable to provide barbed sutures with improved retaining strength or anchoring ability in both directions of the barbed suture. This can enhance the barbed suture's capability to retain tissue with improved wound closure or tissue approximation performance. Current unidirectional barbed sutures available on the market comprises barbs with barb tips oriented in the opposite direction to the insertion end, so that the unidirectional barbed suture can be inserted in the tissue without damaging the tissue. While placed in the tissue, the barb resists the movement of the barbed suture in the direction opposite to the insertion end. The amount of resistance depends on the arrangement and configuration of the barb (i.e. barb length, barb angle, barb thickness, barb material, etc.). However, the suture is still able to move in the insertion direction after the suture is placed in the tissue if there is no stopper at the end of the suture. On the other hand, the bi-directional barbed suture is able to provide suture retaining strength in both directions of the barbed suture. However, two insertion devices (e.g. surgical needles) and more complex surgical procedures are required to place the bi-directional barbed suture in the operation. Therefore, it is desired to have a barbed suture that is easy to use and is capable of providing suture retaining strength in both directions of the barbed suture.

In one embodiment of this invention, the barbed suture 10 comprises a plurality of barbs 17 which are made of substantially V-shaped filaments 12 with a peak and two ends 13, 14 on each filament 12. Both ends 13, 14 of each barb 17 are attached to the main body of the suture thread 11 and spaced apart from each other as illustrated in FIG. 1. For example, in some embodiments, the ends 13, 14 of the barb 17 are separated by a distance equal to or approximately equal to the width of the suture 11 so that the ends 13, 14 of the barbs 17 are disposed on opposing sides of the suture 11. The separated ends 13, 14 serve two purposes: First, the larger base on the suture thread 11 allows better support and strengthens the barb 17. This, in turn, can increase the tissue retention strength for the barbed suture 10. Second, separated ends 13, 14 form a larger space 250 between filament 12 and the suture thread 11 allowing more tissue to grow into that space 250 with higher fixation strength. This enhanced fixation strength comes from the relatively higher force required to break the filament 12 in order to dislodge the barb 17 from the tissue which has grown into and around the filament 12. In addition, the fixation against movement by the ingrown tissue in the barb 17 is capable of resisting suture 10 movement in either direction of the suture thread 11 with improved wound closure or tissue approximation performance. On the other hand, current barbed suture available on the market usually comprises barb with only single attachment point on the suture thread. Even though tissue may grow around the barb, less effort is required to dislodge the barb (without breaking the barb) from the surrounding tissue.

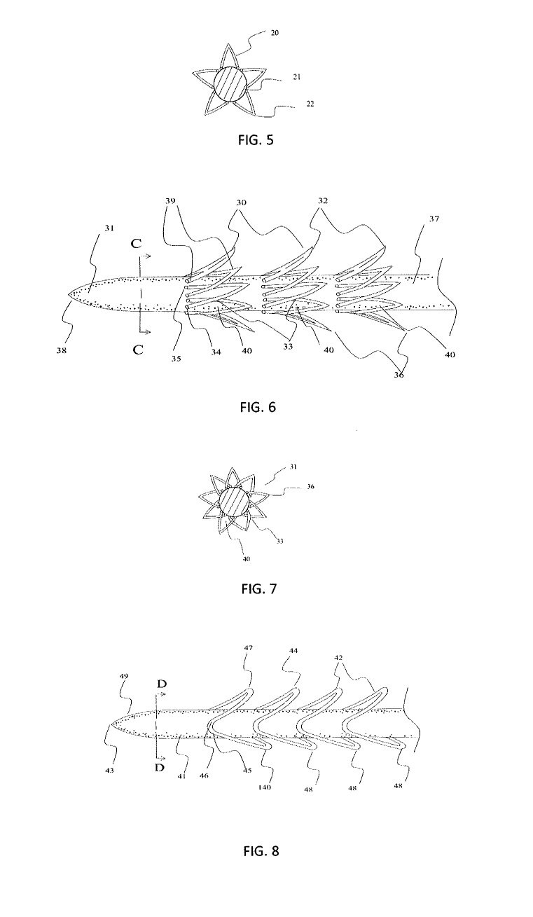

Although a barb 17 with angular barb tip 12 is illustrated in FIGS. 2 and 3, the present invention includes barbed suture of a wide variety of barb and barb tip configurations and their arrangement on the suture thread. FIG. 4 shows a barbed suture 21 with alternative barb 20 and barb tip 22 configuration. In this embodiment, this new unidirectional barbed suture 21 comprises a suture thread 26 and a plurality of barbs 20 pointed away from the insertion direction 27 of the barbed suture 21. Each barb 20 is made of substantially V-shaped filament 25 with a barb tip 22 and two ends 23, 24. Both ends 23, 24 of filament 25 are attached to the main body of the suture thread 26 and spaced apart from each other. In some embodiments, the ends 23, 24 are spaced apart less than the width of the suture 26, such as about ¼, ⅓, ½, ⅔ or 3/3 the width of the suture 26. As shown in FIG. 4, the barb tip 22 is configured to penetrate the tissue (not shown). The cross sectional view of barbed suture 21 from Line B-B is illustrated in FIG. 5. The barbs tips 22 are successively staggered at approximately 72 degree intervals.

In another embodiment of the present invention, a new unidirectional barbed suture 31 comprises a suture thread 37 and a plurality of barbs 30 pointed away from the insertion direction 38 of the barbed suture 31, as shown in FIG. 6. Each barb 30 is made of substantially V-shaped filament 39 with a barb tip 36 and two ends 34, 35. Both ends 34, 35 of filament 39 are attached to the main body of the suture thread 37 and spaced apart from each other. Successive barbs 30 are disposed circumferentially around the suture thread 37 to form a web structure 32. The barbs 30 within each web structure 32 meet or overlap with adjacent barbs 30 at junctions 33. This barbs 30 deposition allows more than one barbs 30 with enhanced retention strength on the same plane perpendicular to the central axis of the suture 37 without reducing the space 40 between the attached ends 34, 35. In yet another embodiment of the present invention, the barbs 30 are connected with successive barbs 30 at junctions 33. The connection of the junctions 33 can be made by typical bonding techniques such as heat, ultrasound, laser, adhesive, etc. The cross sectional view of barbed suture 31 from Line C-C is illustrated in FIG. 7. This interconnected web structure 32 can further enhance the strength of the barbs 30 and their resistance against movement in the direction of the barb tip 36. After the barbed suture 31 is placed in the tissue (not shown), the web structure 32 is allowed to extend radially outwards or “stand up” penetrating the tissue if the suture 31 is pulled in the direction of the barb tip 36. In addition, the web structure 32 comprises spaces 40 between the barbs 30 and suture thread 37 allowing tissue to grow in and around the web structure 32 with enhanced tissue fixation. This enhanced tissue fixation comes from the interconnected web structure 32, and higher force is required to break the web structure 32 in order to dislodge the web structure 32 from the imbedded tissue. In addition, this enhanced tissue fixation acts against suture 31 movement in either direction of the suture thread 37. Therefore this innovated barbed suture 31 enables suture retention in both directions of the suture 31 with improved wound closure or tissue approximation performance.

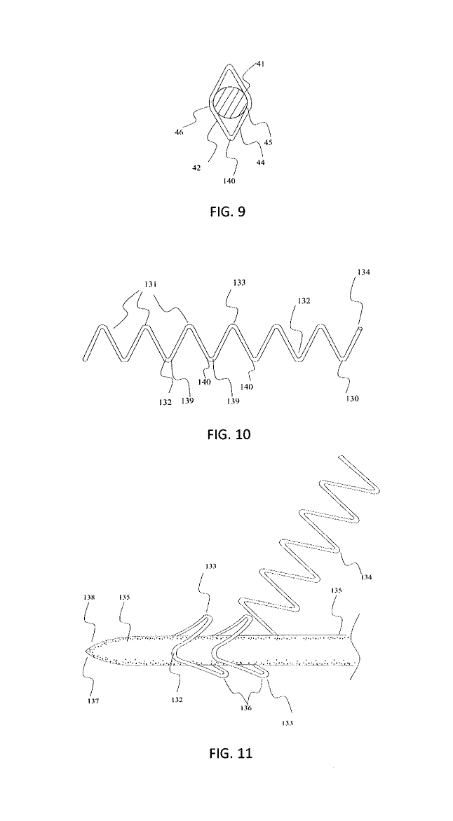

In yet another embodiment of the present invention, a new unidirectional barbed suture 49 comprises a suture thread 41 and a plurality of barbs 42 pointed away from the insertion end 43 of the barbed suture 49 shown in FIG. 8. Each barb 42 is made of substantially V-shaped filament 44 with a peak 140 and two ends 45, 46. Both ends 45, 46 of filament 44 are attached to the main body of the suture thread 41 and spaced apart from each other. Successive barbs 42 are jointed with adjacent barbs 42 at the ends 45, 46 and are disposed spirally around the suture thread 41. In some embodiments, the barbs are formed continuously from a single filament. The peak 140 of the V-shaped filament 44 forms the barb tip 48 which is configured to engage tissue anchoring the barbed suture 49 in the tissue. In addition, the barb tip 48 points away from the insertion end 43 of the suture thread 41 in an acute angle with the central axis of the suture thread 41. The cross sectional view of barbed suture 49 from Line D-D is illustrated in FIG. 9. The barbs 42 are staggered on the peripheral of suture thread 41 at approximately 180 degree intervals.

Even though the successive barbs 42 described above is made by joining the ends 45, 46 of successive V-shaped filaments 44 together, it can also be constructed by many different methods in various configurations. In an exemplary embodiment shown in FIG. 10, a continuous barbs string 134 can be made by a zigzag (or weave, sinuous) filament 130 comprising a plurality of connected substantially V-shaped filaments 131 with peaks 133 and ends 139, 140. The ends 139, 140 are connected to form valleys 132. Then this continuous barbs string 134 is spirally wound on the surface of a suture thread 135 as shown in FIG. 11. The valleys 132 of the continuous barbs string 134 are attached to the suture thread 135. The peaks 133 of the continuous barbs string 134 form the barbs tips 136 and are pointed away from the insertion end 137 of the suture 138 and in an acute angle with the suture thread 135. In some embodiments, the continuous barbs string 134 is formed through extrusion. In other embodiments, the continuous barbs string 134 can be formed by cutting a sheet.

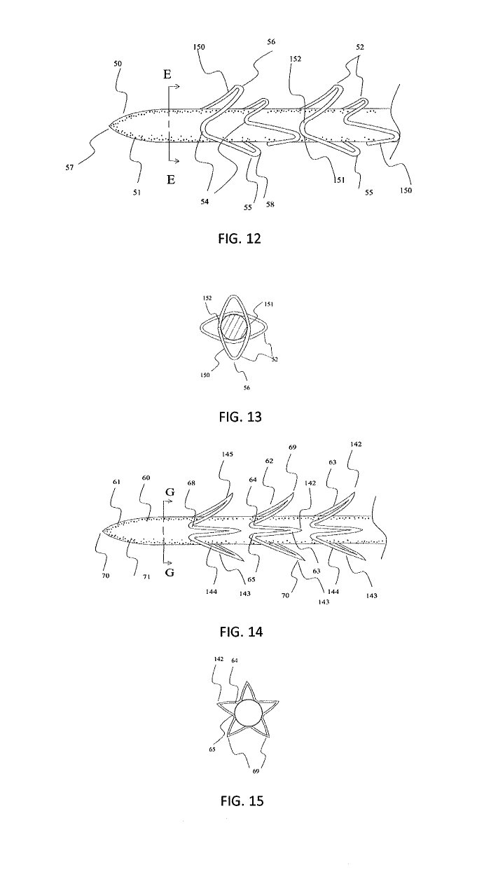

In further another embodiment of the present invention, an alternative method to make barbed suture is disclosed. An unidirectional barbed suture 50 comprises a suture thread 51, and a plurality of barbs 52 attached to said suture thread 51 as shown in FIG. 12. Each barb 52 is made of substantially V-shaped filament 150 with a peak 55 and two ends 151, 152. In some embodiments, the barb lengths can alternate between relatively long barbs and relatively short barbs. Both ends 151, 152 of filament 150 are attached to the main body of the suture thread 51 and spaced apart from each other. The peaks 55 of the substantially V-shaped filament 150 form the barbs tips 56 and are pointing away from the insertion end 57 of the suture 50 in an acute angle with the suture thread 51. Successive barbs 52 are joined with adjacent barbs 52 at the ends 151, 152 and are disposed spirally around the suture thread 51. FIG. 13 is the cross sectional view of barbed suture 50 from Line E-E. The barbs tips 56 are spirally wound and arranged on the suture thread 51 surface 90 degree apart from each other.

Alternatively, a continuous barbs string 58 can be made by a zigzag (or weave, sinuous) filament comprising a plurality of connected substantially V-shaped filaments 150 with peaks 55 and ends 151, 152. The ends 151, 152 of successive filaments 150 are connected to form valleys 54 of the continuous barbs string 58. Then the continuous barbs string 58 is spirally wound and arranged on the surface of suture thread 51. The valleys 54 of the continuous barbs string 58 are attached to the suture thread 51. The peaks 55 of the continuous barbs string 58 form the barbs tips 56 and are pointing away from the insertion end 57 of the suture 50 in an acute angle with the suture thread 51.

Although barbed sutures with some exemplary barbs disposition patterns on the suture thread are illustrated above, the present invention includes barbed suture of a wide variety of barb configurations and their arrangement on the suture thread. In one embodiment of the present invention, an unidirectional barbed suture 60 comprises a suture thread 61, and a plurality of barbs 62 attached to said suture thread 61 as shown in FIG. 14. Each barb 62 is made of substantially V-shaped filament 63 with a peak 142 and two ends 64, 65. Both ends 64, 65 of filament 63 are attached to the main body of the suture thread 61 and spaced apart from each other. The peaks 142 of the substantially V-shaped filament 63 form the barbs tips 69 and are pointed away from the insertion end 70 of the suture 60 in an acute angle with the suture thread 61. Successive barbs 62 are jointed with adjacent barbs 62 at the ends 64, 65 and are disposed circumferentially around the suture thread 61 to form a barb circle 143 on the surface of suture thread 61. There is a predetermined distance between successive barb circles 143 along the length of the barbed suture 60. FIG. 15 is the cross sectional view of barbed suture 60 from Line G-G. The barbs tips 69 are deposited on the suture thread 61 surface approximately 72 degree apart from each other.

Alternatively, a plurality of V-shaped filaments 63 are connected at the ends 64, 65 to form a continuous barbs string 145 before it is attached to the suture thread 61. The continuous barbs string 145 comprises a plurality of connected V-shaped filaments 63 having peak 142 and valleys 68 which are formed by connecting the ends 64, 65. The continuous barbs string 145 with valleys 68 and peaks 142 is wound circumferentially to form a barb circle 143 on the surface of suture thread 61. The valleys 68 of the continuous barbs string 143 are attached to the suture thread 61, and the peaks 142 of the continuous barbs string 143 form the barbs tips 69. There is a pre-determined distance between successive barb circles 143 along the length of the suture thread 61.

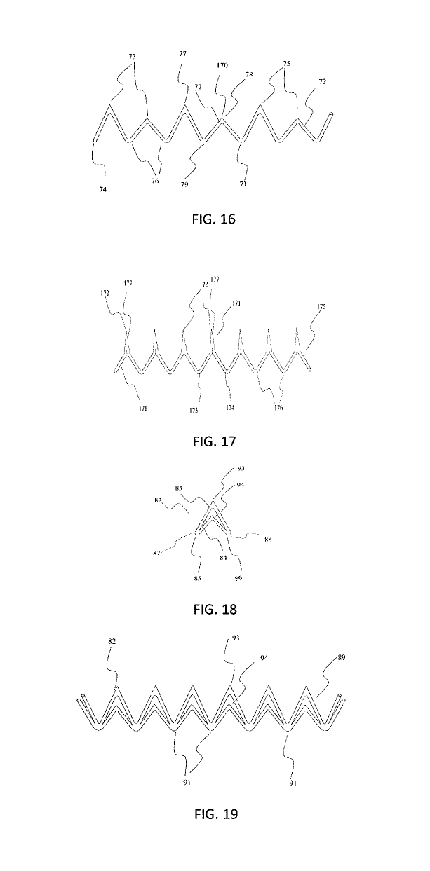

Other examples for barb configurations are illustrated in FIGS. 16, 17. In FIG. 16, each barb 72 is made of substantially V-shaped filament 73 having a peak 170 and two ends 71, 79. A plurality of V-shaped filaments 73 are connected at the ends 71, 79 to form a continuous barbs string 74 with a plurality of peaks 170 and valleys 76 as shown in FIG. 16. The height of the filaments 73 varies and alternates between tall filament 77 and short filament 78. Then this continuous barbs string 74 is wound spirally or circumferentially on the surface of suture thread. The valleys 76 of the continuous barbs string 74 are attached to the suture thread. The peaks 170 of the continuous barbs string 74 form the barbs tips. Because there are both tall filament 77 and short filament 78, the resulting barbed suture comprises barbs with various lengths on the suture thread. Many of the barb embodiments described herein, even if not described or illustrated as having varying barb lengths, may be constructed with varying filament lengths to generate barbs of varying lengths.

In FIG. 17, each barb 177 is made of substantially V-shaped filament 171 with a peak 172 and two ends 173, 174. The tip of the peak 172 is shaped like needle to penetrate tissue. Individual barb 177 can be attached to the suture thread. In this case, both ends 173, 174 of filament 171 are attached to the main body of the suture thread and spaced apart from each other. Alternatively, a plurality of V-shaped filaments 171 are connected at the ends 173, 174 to form a continuous barbs string 175 with a plurality of peaks 172 and valleys 176. The continuous barbs string 175 is wound spirally or circumferentially on the surface of suture thread. The valleys 176 of the continuous barbs string 175 are attached to the suture thread. The peaks 172 of the continuous barbs string 175 form the barbs tips.

In further another embodiment of the present invention, an alternative method to make barb and barbed suture is disclosed. Each barb 82 comprises two overlapping V-shaped filaments 83, 84 of two different heights as shown in FIG. 18. As a consequence, there are two peaks 93, 94 for each barb 82 with enhanced anchoring strength. The ends 85, 86 of V-shaped filaments 83 are joined with the ends 87, 88 of V-shaped filaments 84 respectively. Individual barb 82 can be attached to the suture thread in this disclosure. In this case, joined ends 85, 86, 87, 88 of filaments 83, 84 are attached to the main body of the suture thread and spaced apart from each other. Alternatively, a plurality of successive barbs 82 are connected at the ends 85, 86, 87, 88 to form a continuous barbs string 89 with a plurality of peaks 93, 94 and valleys 91 as shown in FIG. 19. The continuous barbs string 89 is wound spirally or circumferentially on the surface of suture thread. The valleys 91 of the continuous barbs string 89 are attached to the suture thread. The peaks 93, 94 of the continuous barbs string 89 form the barbs tips.

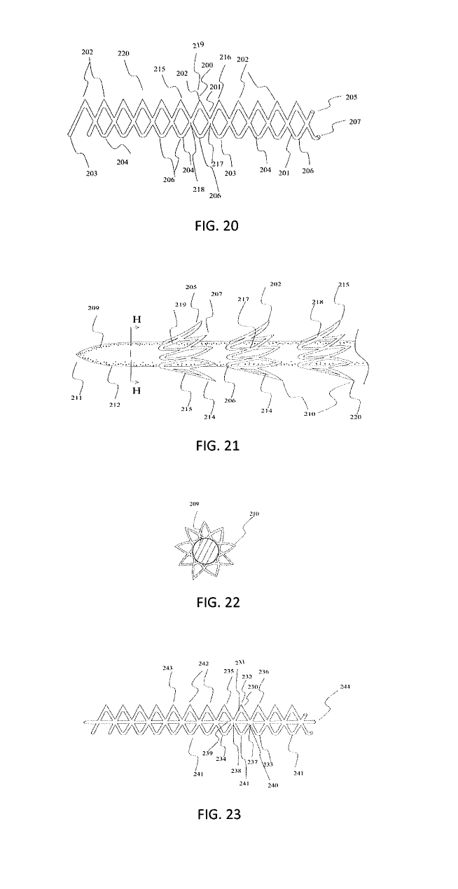

In further another embodiment of the present invention, an alternative method to make barbs and barbed suture is disclosed. In FIG. 20, each barb 200 is made of substantially V-shaped filament 201 with a peak 202 and two ends 203, 204. It overlaps with two adjacent filaments 215, 216 at junctions 217, 218 to form a lattice unit 219. A plurality of lattice units 219 are connected at the ends 203, 204 to form a lattice structure 220 with a plurality of peaks 202 and valleys 206 as shown in FIG. 20. In some embodiments, the lattice units 219 can be diamond shaped. Then this lattice structure 220 is wound circumferentially on the surface of suture thread 209 to form a barb circle 215 as shown in FIG. 21. The valley 206 is attached to the suture thread 209. The peaks 202 of the lattice structure 220 form the barbs tips 210 and are pointed away from the insertion end 211 of the barbed suture 212 and in an acute angle with the suture thread 209. There is a pre-determined distance between successive barb circles 215 alone the length of the suture thread 209. FIG. 22 is the cross sectional view of barbed suture 212 from Line H-H. The barbs tips 210 are arranged on the suture thread 209 surface approximately 40 degree apart from each other. Alternatively, the junctions 217, 218 of the lattice structure 220 are connected. The binding methods are selected from heat, ultrasound, laser, adhesive or other binding technique. Alternatively, the lattice structure 220 is wound spirally on the surface of suture thread 209.

In yet another embodiment of the present invention, an alternative method to make barb and barbed suture is disclosed. In FIG. 23, each barb 230 is made of substantially V-shaped filament 231 with a peak 232 and two ends 233, 234. It overlaps with two adjacent filaments 235, 236 at junctions 237, 238. Then the junctions 237, 238 are connected with a filament 239 to form a lattice unit 240 with valley 241 and peak 242 as shown in FIG. 23. In some embodiments, the lattice unit is diamond shaped and bisected by the filament into two triangular subunits. The valley 241 is attached to the suture thread. The peak 242 of the lattice unit 240 forms the barb tip 243 and is pointed away from the insertion end of the barbed suture and in an acute angle with the suture thread. Alternatively, a plurality of lattice units 240 are connected at the ends 233, 234 and junctions 237, 238 to form a lattice structure 244 with a plurality of peaks 242 and valleys 241. Then this lattice structure 244 is wound circumferentially or spirally on the surface of suture thread. The valleys 241 are attached to the suture thread. The peaks 242 of the lattice unit 240 form the barb tip 243.

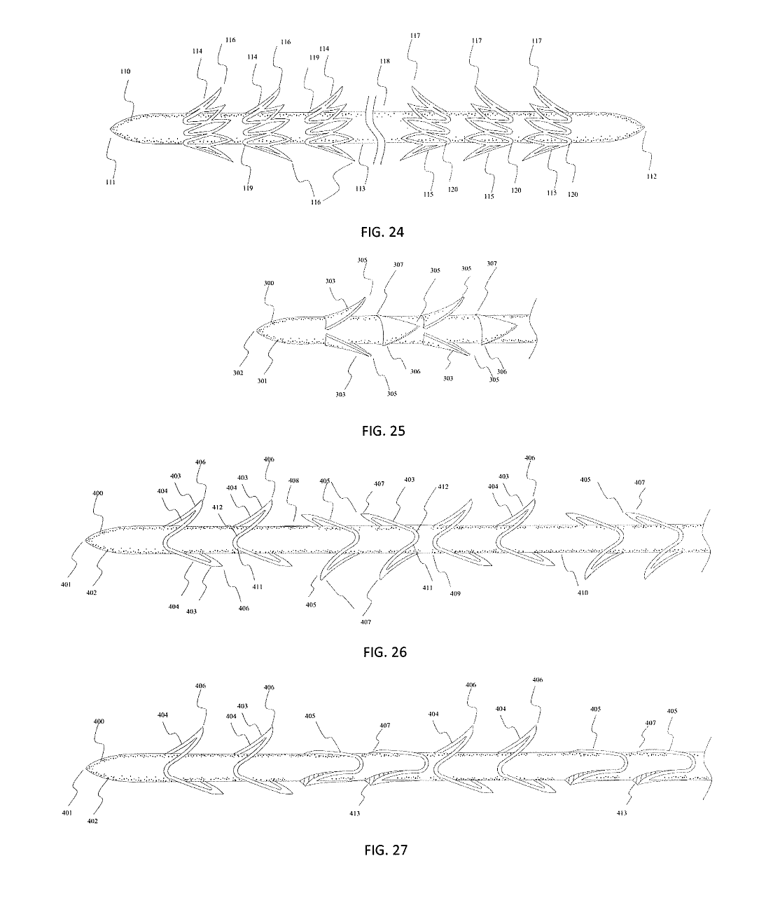

In yet another embodiment of the present invention, the barbed suture can be bidirectional as shown in FIG. 24. This bidirectional barbed suture 110 comprises a suture thread 113, two insertion ends 111, 112, and a plurality of barbs 114, 115 attached to said suture thread 113. The pluralities of barbs 114, 115 are arranged on said suture thread 113 in two separate sections. In the first section, the barbs 114 are bound to the suture thread 113 at valleys 119, and comprise barbs tips 116 pointed away from the insertion points 111. With this configuration, this section of the barbed suture 110 can be inserted in the tissue in the direction of insertion points 111, but resists movement in the opposite direction of insertion point 111. If the barbed suture 110 is pulled in the opposite direction of insertion points 111, the tips 116 penetrate the tissue (not shown) and the barbs 114 extend radially outwards or “stand up” to increase the cross sectional profile of the barbed suture 110. In the other section of the barbed suture 110, the barbs 115 are bound to the suture thread 113 at valleys 120, and comprise barbs tips 117 pointed away from the insertion points 112. With this configuration, this section of the barbed suture 110 can be inserted in the tissue in the direction of insertion points 112, but resists movement in the opposite direction of insertion point 112. At transition point 118, the barb tips 116, 117 reverse about 180 degree. The bidirectional barbed suture is designed to enhance the retaining strength of the suture in the tissue in both directions of the suture thread.

In yet another embodiment of the present invention, an alternative method to make barb and barbed suture is disclosed. In FIG. 25, the barbed suture 300 comprises a suture thread 301, insertion end 302, and a plurality of barbs 303 attached to said suture thread 301. Each barb 303 is made of substantially V-shaped sheet 304 with a barb tip 305 and two ends 306, 307. The barb 303 is bound to the suture thread 301 at ends 306, 307, and comprise barb tip 305 pointed away from the insertion end 302. With this configuration, this barbed suture 300 can be inserted in the tissue in the direction of insertion end 302, but resists movement in the opposite direction of insertion end 302. If the barbed suture 300 is pulled in the opposite direction of insertion end 302, the tips 305 penetrate the tissue (not shown) and the barbs 303 “stand up” or extend radially outwards to increase the cross sectional profile of the barbed suture 300.

In addition to those barb parameters discussed above, the spatial arrangement of the barbs and barbs tips on the suture thread is also important to the retaining strength of the barbed suture in the tissue. In contrast to traditional sutures, the barbed suture can spread out the retention load along the length of the suture thread evenly. By optimizing the arrangement of barbs and barbs tips on the suture, the retaining strength of the barbed suture should therefore be enhanced with improved tissue holding and wound closure strength. In one embodiment of the present invention, the arrangement of the barbs tips on the suture thread may be staggered circumferentially, staggered axially, staggered both axially and circumferentially, twisted with single spiral, twisted with multiple spirals, overlapped, random or combinations thereof.

For example, in some embodiments, the arrangement of the barbs tips 15 on the suture thread 11 may be staggered circumferentially as shown in FIGS. 1, 4 and 6, this allows multiple barbs tips on each plane perpendicular to the suture axis without reducing suture cross-section or losing suture strength. On the other hand, the suture cross-section and strength will be reduced if the barb is formed by cutting into the suture thread. As a consequence, no more than one barb is cut on each plane perpendicular to the suture axis to avoid losing suture strength. In one embodiment of the present invention, the cross-section of the suture thread is substantially round, and the barbs tips are staggered circumferentially within the range of 0 to 180 degree intervals.

In another embodiment, the arrangement of the barbs tips on the suture thread may be staggered axially along a length of the suture thread as shown in FIG. 9, this arrangement allows less cross section area on each plane perpendicular to the suture axis, thereby a reduced suture pulling effort through the tissue. In one embodiment, the successive barbs tips may be formed at a predetermined distance between adjacent barbs along a length of the suture thread. The predetermined distance is substantially within the range of 0 to 2.0 cm, and may further be approximately 0.2 cm.

In yet another embodiment, the arrangement of the barbs tips on the suture thread may be staggered both circumferentially and axially spaced with a distance between successive barbs as shown in FIG. 12, this arrangement allows the optimization of the retaining strength and suture pulling force through the tissue. The predetermined distance between the axially spaced barbs is substantially within the range of 0 to 2.0 cm, and may further be approximately 0.1 cm.

In further another embodiment, the arrangement of the barbs tips on the suture thread may be twisted with single or multiple spirals on the suture surface. This arrangement enhances the chance for the barbs to engage tissue effectively with a higher retaining strength especially in the soft tissue with fewer connective fibers. In the other embodiment, the arrangement of the barbs on the suture thread may be overlapped with successive barbs as shown in FIGS. 6 and 21, this arrangement increases the retaining strength and suture pulling force through multiple overlapped barbs on the suture.

In yet another embodiment, a barbs circle is formed by successive barbs 20 around the circumference of the suture thread 26 as shown in FIG. 4. A plurality of axially spaced barb circles is disposed along the length of the suture 21 with a distance between them. The predetermined distance between adjacent circles is substantially within the range of 0 to 2.0 cm, and may further be approximately 0.1 cm. In yet another embodiment, the barbs are arranged in a way so that at least two adjacent barbs are disposed with one barb overlapping on the other as shown in FIG. 6. In yet another embodiment, the barbs are disposed randomly on the exterior surface of the suture thread.

One of the limitations on the current bidirectional barbed suture is the relatively more complex and/or difficult method to deliver the bidirectional barbed suture in the tissue. For traditional bidirectional barbed suture with one transitional segment, it comprises two needles with one needle at each end of the suture thread. Each needle is linked with a section of barbed suture with barb tips pointed away from the needle until the transition segment is reached. At the transition segment, the configuration of the barbs changes and the direction of the barb tip reverse about 180 degree. As a consequence, each needle with its accompanied section of barbed suture can only advance in the tissue until the transition segment is reached. If the suture is pulled pass the transition segment, the resistance against pulling increases because the barb tips have reversed direction pointing toward the pulling direction. After the suture is inserted in the tissue, because the transition segment comprises barbs tips pointed to both directions of the suture thread, it resists against moving in both directions and thus serves as an anchor of the suture in the tissue. As a consequence, the bidirectional barbed suture can improve retaining strength or anchoring ability in both directions of the barbed suture. More transition segment along the suture means higher retaining strength in both directions of the barbed suture in the tissue. However, the more complex two needles system described above is required to deliver bidirectional barbed suture with one transition segment, and it can't be used to deliver bidirectional barbed suture with more than one transition segments as shown in FIG. 26. A relatively inconvenient delivery device such as a sheath is usually needed to temporarily cover the barbs in order to place this type of suture in the tissue. As a result, it would be desirable to provide barbed sutures with improved retaining strength or anchoring ability in both directions of the barbed suture without the need for complex delivery device such as sheath. This can enhance the barbed suture's ability to hold tissue together with improved wound closure or tissue approximation performance and allows the barbed suture to be readily deployed in the tissue without damaging the tissue.

To address this issue, the bidirectional barbed suture of the present invention also comprises a binder which is water soluble or degradable in the body. For those barbs with tips directed to the insertion end, the binder is able to temporarily bind those barbs to the main body of the suture thread to maintain or reduce the cross-sectional profile of the barbed suture, thereby reducing resistance against insertion during the insertion of the barbed suture in the tissue. As a result, the barbed suture is allowed to be readily deployed in the tissue without damaging the surrounding tissue. After the insertion, this binder will be dissolved by body fluid or degraded in the body releasing the barbs on the suture to anchor the barbed suture in the tissue.

In one embodiment of this invention, the water soluble and/or degradable binder can be applied to all the barbs of the suture, and because the water soluble or/and degradable binder is effective in binding all the barbs to the suture thread and reducing friction for barbs pointed in either direction of the bidirectional barbed suture, barbed suture with more than one transition segments can be inserted into the tissue. After the barbed suture is inserted in the body, the binder will be dissolved by body fluid or degraded in the body releasing the barbs on the suture. Those released barbs can resist suture movement in the same direction as the barb tips. As a consequence, the bidirectional barbed suture in this invention comprises higher retaining strength in both directions of the barbed suture and can be readily delivered in the tissue.

For example, in some embodiments, as shown in FIG. 26, a bidirectional barbed suture system 400 comprises an insertion end 401, a suture thread 402, and a plurality of barbs 404, 405 attached to said suture thread 402. Each barb 404 is made of substantially V-shaped filament 403 with a barb tip 406 and two ends 411, 412 bound to the suture thread 402. The said pluralities of barbs 404, 405 are arranged on said suture thread 402 in various patterns. Some of the barbs 404 have tips 406 pointed away from the insertion end 401. With this configuration, the barbs 404 can be inserted in the direction of insertion end 401 without penetrating the tissue or causing high resistance during insertion, but resists movement in the opposite direction of insertion end 401. On the other hand, some of the barbs 405 have tips 407 pointed toward the insertion end 401. At transition points 408, 409, 410, the barb tips 406, 407 reverse direction. For those barbs 405 having tips 407 pointed toward the insertion end 401, they will penetrate the tissue or “stand up” or extend radially outwards, thereby increasing the cross-sectional profile (and resistance) of the barbed suture 400 when the suture 400 is pulled through the tissue. The resistance caused by this tissue penetrating or “stand up” barbs 405 is the major mechanism for the retaining of the suture 400 in the tissue.

At transition segments 408, 409, 410, they comprise barbs 405 with tips 407 resisting movement in the direction of insertion 401 and barbs 404 with tips 406 resisting movement in the opposite direction of insertion 401. As a consequence, transition segments 408, 409, 410, act as “stops” or “anchors” for the suture 400 against movement in both directions of the suture thread 402, and thereby providing barbed suture with high retaining strength in both directions of the suture thread 402.

However, this resistance caused by tissue penetrating or “stand up” barbs 405 in the insertion direction 401 needs to be minimized during the insertion of the suture 400 in the tissue. This high resistance not only increases suture 400 pulling force, but also causes damage in the surrounding tissue. Because there are more than one transition segments 408, 409, 410, traditional two needles delivery method can't be used to deliver the barbed suture 400 in the tissue. As a consequence, a more complex and inconvenient delivery system such as a sheath or a hypotube is usually required to temporarily cover the barbs 405 during the delivery of the suture 400 in the tissue.

In one embodiment of the present invention, an alternative method to make barb and bi-directional barbed suture is disclosed. In FIG. 27, those barbs 405 having tips 407 pointed toward the insertion end 401 are temporarily bound to the suture thread 402 with a binder 413 to prevent the tips 407 from penetrating the tissue (not shown), “standing up” or extending radially outwards, thereby decreasing the resistance during the insertion of the suture 400 into the tissue. As a consequence, the bi-directional barbed suture 400 is allowed to move through tissue and be inserted into the tissue in the direction of insertion end 401.

As soon as the suture 400 is placed in the tissue, those un-bound barbs 404 having tips 406 pointed away from the insertion end 401 can provide suture instantaneous retaining strength opposite to the insertion end 401. If the suture 400 is pulled in the direction opposite to the insertion end 401, the tips 406 penetrate the tissue (not shown) and the barbs 404 “stand up” or extend radially outwards to raise the cross sectional profile of the suture 400, thereby increasing the resistance against pulling in the direction opposite to the insertion end 401. After the binder 413 on the barbs 405 is dissolved by body fluid or degraded in the body, those barbs 405 with tips 407 pointed toward the insertion end 401 are released providing retaining strength toward insertion direction 401 as shown in FIG. 26. If the suture 400 is pulled in the direction of insertion end 401, the tips 407 penetrate the tissue (not shown) and the barbs 405 “stand up” or extend radially outwards to raise the cross sectional profile of the suture 400, thereby increasing the resistance against pulling in the direction of the insertion end 401. As a result, the bidirectional barbed suture 400 of this invention comprises retaining strength in both directions of the suture thread 401 and can be delivered in the tissue readily without the need for complex delivery system such as a sheath or a hypotube.

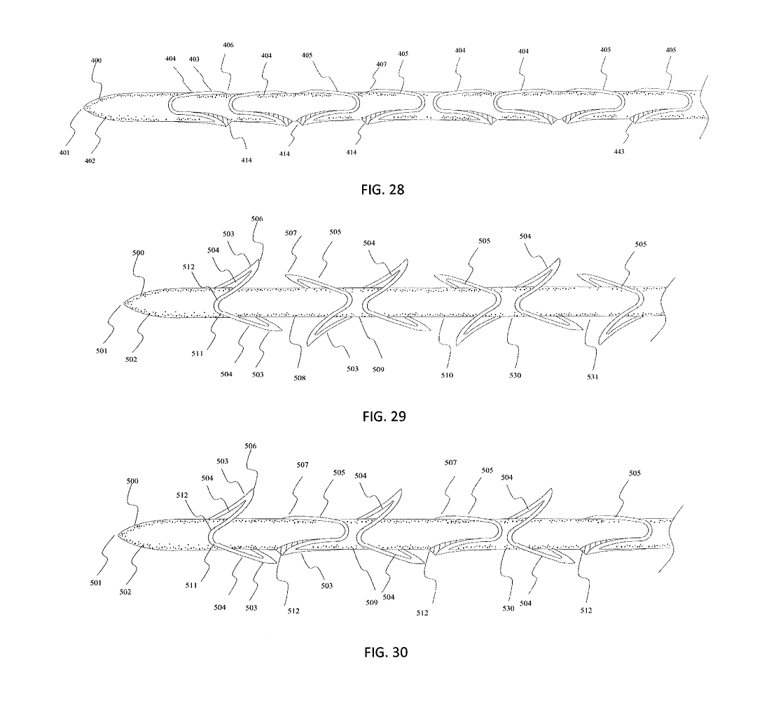

In the other embodiment of the present invention, the binder is effective in binding or covering the barbs to the suture thread regardless the barb tips orientation (either unidirectional and bidirectional). As illustrated in FIG. 26, a bidirectional barbed suture system 400 comprises a plurality of barbs 404, 405 attached to said suture thread 402. Each barb 404, 405 is made of substantially V-shaped filament 403 with a barb tip and two ends 411, 412 bound to the suture thread 402. The said pluralities of barbs 404, 405 are arranged on said suture thread 402 in various patterns. Some of the barbs 404 have tips 406 pointed away from the insertion end 401. On the other hand, some of the barbs 405 have tips 407 pointed toward the insertion end 401. All the barbs 404, 405 are bound by said binder 414 regardless the tip orientation as shown in FIG. 28. The binder 414 prevents the tips 406, 407 from penetrating the tissue (not shown) or “standing up” to raise the cross sectional profile of the suture 400, thereby reducing resistance during the insertion of the suture 400 into the tissue. As a consequence, the suture 400 can be inserted into the tissue in the direction of insertion 401. In the other embodiment of the present invention, because the binder 414 is effective in binding the barbs 404, 405 to the suture thread 402 regardless the tip orientation, the barbed suture 400 can be inserted into the tissue from either end of the suture 400. Alternatively, the barbed suture is allowed to be inserted in the tissue from both ends of the suture thread 402. In another embodiment of the present invention, this suture is a unidirectional barbed suture with barb tips pointed to one end of the suture. Because the binder is effective in binding or covering most of the barbs to the suture thread regardless the barb tips orientation (either toward or away from the insertion end), the unidirectional barbed suture can be inserted into the tissue from either end of the suture. Alternatively, the unidirectional barbed suture is allowed to be inserted in the tissue from both ends of the suture thread.

After the bidirectional barbed suture 400 is inserted into the tissue, the binder 414 is dissolved or degraded in the body. Without the restraint by the binder 414, the barbs 404, 405 are released from the suture thread 402 and extend radially outwards as shown in FIG. 26. Those barbs 405 with tips 407 pointed toward the insertion end 401 are released providing retaining strength toward insertion direction 401. On the other hand, those barbs 404 with tips 406 pointed opposite to the insertion end 401 are released providing retaining strength in a direction opposite to the insertion direction 401. As a result, the bidirectional barbed suture 400 of this invention comprises retaining strength in both directions of the suture thread 402 and can be delivered in the tissue readily without the need for more complex delivery system such as sheath or hypotube.

In yet the other embodiment of the present invention, the barbed suture further comprises a delivery device that is linked to the insertion end of the barbed suture to assist the insertion of the suture in the tissue. The said delivery device may be a needle. Because the water soluble or/and degradable binder is effective in restraining barbs and reduces friction for barbs pointed in either direction of the barbed suture, the needle can be linked at either end of the barbed suture. In another embodiment of the present invention, the barbed suture further comprises two delivery devices that are linked to both ends of the barbed suture. The said insertion devices are needles. Because the water soluble or/and degradable binder is effective in restraining barbs and reduces friction for barbs pointed in either direction of the barbed suture, both needles can be used for inserting the suture into the tissue.

For another example of some embodiments in this invention, as illustrated in FIG. 29, a bidirectional barbed suture system 500 comprises a plurality of barbs 504, 505 attached to said suture thread 502, and a plurality of transition segments 508, 509, 510, 530, 531. Each barb 504, 505 is made of substantially V-shaped filament 503 with a barb tip and two ends 511, 512 bound to the suture thread 502. The said pluralities of barbs 504, 505 are arranged on said suture thread 502 in various patterns. Some of the barbs 504 have tips 506 pointed away from the insertion end 501. On the other hand, some of the barbs 505 have tips 507 pointed toward the insertion end 501. At transition segments 508, 509, 510, 530, 531, they comprise barbs 505 with tips 507 resisting movement in the direction of insertion direction 501 and barbs 504 with tips 506 resisting movement in the opposite direction of insertion direction 501. As a consequence, transition segments 508, 509, 510, 530, 531 act as “stops” or “anchors” for the suture 500 against movement in both directions of the suture thread 502, thereby increasing retaining strength for the barbed suture 500 in both directions of the suture thread 502. However, current two-needle delivery system can't deliver this bidirectional barbed suture 500 with more than one transition segments.

As shown in FIG. 30, the barbs 505 with tips 507 pointed toward the insertion end 501 are bound to the suture thread 502 by the binder 512. The binder 512 prevents the tips 505 from penetrating the tissue (not shown) or extending radially outwards or “standing up” to raise the cross sectional profile of the suture 500, thereby reducing resistance during the insertion of the suture 500 into the tissue. As a consequence, the bidirectional barbed suture thread 500 with more than one transition segments 508, 509, 510, 530, 531 can be inserted into the tissue in the direction of insertion 501 without the need for sheath or hypotube to cover the barbs 504, 505.

After the bidirectional barbed suture 500 is inserted into the tissue, the binder 512 is dissolved or degraded in the body. Without the restraint by the binder 512, the barbs 505 are released from the suture thread 502 and extend radially outwards as shown in FIG. 29. Those barbs 505 with tips 507 pointed toward the insertion end 501 are released providing retaining strength toward insertion direction 501. On the other hand, those barbs 504 with tips 506 pointed opposite to the insertion end 501 provide retaining strength opposite to insertion direction 501. As a result, the bidirectional barbed suture 500 of this invention comprises retaining strength in both directions of the suture thread 502 and can be delivered in the tissue readily without the need for sheath or hypotube. Alternatively, the binder 512 binds all the barbs 504, 505 to the suture thread 502 and restrains the barbs 504, 505 in both directions of the bidirectional barbed suture 500. As a result, the bidirectional barbed suture 500 with more than one transition segments can be inserted into the tissue from either end of the suture 500.

According to further embodiment of the present invention, the present invention provides barbed suture with a binder which is water soluble or degradable. The binder can be placed between the barb and the main suture thread to restrain the movement and position of the barb as shown in FIG. 30. Alternatively, the binder can be placed on top of the barb to cover and restrain the movement of the barb. In another embodiment, the binder can be placed both on top of the barb, and between the barb and the main suture thread. All those methods serve to temporarily cover or restrain the barb to prevent the barb from penetrating the tissue, to maintain or reduce the cross-sectional profile of the barbed suture and its resistance against needle pulling during the insertion of the barbed suture in the tissue. This binder will be dissolved by body fluid or degraded in the body after the insertion releasing the barbs on the suture to anchor the barbed suture in the tissue. Alternatively, the binder is both water soluble and degradable. After the barbed suture is inserted in the tissue, this binder will be dissolved by body fluid and degraded in the body releasing the barbs on the suture to anchor the barbed suture in the tissue.

Many biocompatible materials can be used as the binder in this invention. In one embodiment of this invention, this biocompatible binder is soluble in water or body fluid. The binder is able to temporarily bind the barb with the main body of the suture thread. It can be applied on the barbed suture by coating, spraying, brushing or dipping with or without solvent. In some embodiments, the binder may be formed into a membrane, ribbon, or sleeve that can be wrapped around one or more barbs. If a solvent is used, the binder is dissolved in the solvent and then applied on the suture. Thereafter, the barbs are pressed on the suture thread to squeeze out excess binder from the interface. Sufficient pressure should be maintained on the barbs when the solvent is evaporating to ensure good binding between the barb and the suture thread. After the barbed suture is inserted in the tissue, the binder is dissolved in the body fluid to release the barb on the suture anchoring the suture in the tissue. In one embodiment, the release rate of the barb and the solubility of the binder in water or body fluid can be controlled by tailoring the composition, hydrophilicity, and the solubility of the binder. Binder with more hydrophilicity usually dissolves faster in the body fluid. The examples of water soluble binder include saccharides and their derivatives, protein, surfactants, adhesives, gums, water soluble polymers, minerals, etc. Examples of surfactants are Cetalkonium chloride, alkoxylated surfactant which includes, but are not limited to, alkylphenol ethoxylates, ethoxylated fatty acids, alcohol ethoxylates, alcohol alkoxylates, polysorbate, Tween, and ethylene oxide-propylene oxide copolymers (Poloxamers, Tetronics or Pluronics) and mixtures thereof. Examples of saccharides are monosaccharides (e.g. glucose, fructose, galactose, etc.), disaccharides (e.g. sucrose, maltose, lactose, etc.), polysaccharides (e.g. starches, modified starch, pregelatinized starch, cellulose or modified cellulose, methyl cellulose, ethyl cellulose, ethyl methyl cellulose, hydroxyethyl cellulose, hydroxyethyl methylcellulose, hydroxyl propyl methylcellulose, ethyl hydroxyl ethyl cellulose, microcrystalline cellulose, sodium carboxymethyl cellulose, hydroxypropyl cellulose, carboxymethyl cellulose, cellulose ethers, Hypromellose, etc.), sugar alcohols (e.g. xylitol, sorbitol or maltitol, glycerol, erythritol, threitol, arabitol, ribitol, mannitol, galacitol, fucitol, etc.) and mixtures thereof. Examples of proteins are gelatin, collagen, zein, etc. Examples of minerals are calcium phosphates (e.g. monocalcium phosphate, dicalcium phosphate, tricalcium phosphate, hydroxyapatite, apatite, octacalcium phosphate, biphasiccalcium phosphate, tetracalcium phosphate, amorphous calcium phosphate, etc.) and mixtures thereof. Examples of gum are agar, alginic acid, sodium alginate, carrageenan, acacia, gum Arabic, gum copal, kauri gum, spruce gum, gum ghatti, Tragacanth, gum tragacanth, karaya gum, guar gum, locust bean gum, chicle gum, dammar gum, mastic gum, Xanthan gum, etc. and mixtures thereof. Many water soluble polymers can be used as the binder in this invention. The examples of this embodiment include biodegradable and non-biodegradable polymers. They do not cause toxic or inflammatory effects. Examples of water soluble polymers are partially deacetylated polyvinyl alcohol, ethylene-vinyl alcohol copolymers, poly-3-hydroxybutyrate, cellulose acetates, hydrogels, polyvinyl pyrrolidone (PVP) polymer or copolymer, copolyvidone, polyethylene oxide (PEO) polymer or copolymer, poly(ethylene glycol), poly(propylene glycol), polytetramethylene oxide, poly vinyl alcohol (PVA) polymer or copolymer, polymethacrylate, Carbopol, Poloxamer, Tetronics, polyhema polymer or copolymer, Hypan polymer or copolymer, poly(hydroxy ethyl acrylate), poly(hydroxy ethyl methacrylate), hydroxy ethyl cellulose, hydroxy propyl cellulose, hydroxypropyl methylcellulose, hypromellose, methylcellulose, collagen, hyaluronic acid, poly amino acids, fibrin, gelatin, alginate and mixtures thereof.

In one embodiment of this invention, this binder is degradable in body. The binder is able to temporarily bind the barb to the main body of the suture thread. It can be applied on the barbed suture by coating, spraying, brushing or dipping with or without solvent. In some embodiments, the binder may be formed into a membrane, ribbon, or sleeve that can be wrapped around one or more barbs. If a solvent is used, the binder is dissolved in the solvent and then applied on the suture. Then, the solvent will evaporate leaving the binder on the suture. After the barbed suture is inserted in the tissue, the binder is degradable in body to release the barb on the suture anchoring the suture in the tissue. The degradable binder can be substantially, if not completely, degraded, excreted or metabolized by the body. This process may typically take up to several days and is regulated by variables in the formulation and manufacturing of the binder disclosed herein. The degradation by-products may be mainly expelled via normal respiration and excretion. The examples of degradable binder include Candelilla wax, Carnuba wax, degradable polymers, etc. Examples of degradable polymers are polylactic acid polymer or copolymer, polyglycolic acid polymer or copolymer, copolymers of polylactic acid and polyglycolic acid, polyepsilon caprolactone polymer or copolymer, poly caprolactone polymer or copolymer, polyhydroxy butyric acid polymer or copolymer, poly-3-hydroxybutyrate polymer or copolymer, polyorthoesters polymer or copolymer, polyacetals polymer or copolymer, polydihydropyrans polymer or copolymer, poly trimethylene carbonate, collagen, hydrogel, hyaluronic acid, polyacrylamine, poly(vinylcarboxylic acid), polymethacrylic acid, polyacrylic acid polymer or copolymer, poly amino acids, fibrin, chitosan, gelatin, alginate, cellulose acetate phthalate, cellulosic, polylysine, polyarginine, poly aspartic acid, poly glutamic acid, methylcellulose, hydroxy ethyl cellulose, hydroxy propyl cellulose, hydroxypropyl methylcellulose, cellulose acetate, nitrocellulose, hypromellose, cellulose butyrate, polyhydroxyalkanoates, polyhydroxyvalerate (PHV) and polyhydroxyhexanoate, polyanhydrides, polyethylene terephthalate, polyhydroxurethanes, poly(2-hydroxyethyl-methacrylate), polyphosphazenes, polyphosphoesters, polyglactin, catgut, silk, poliglecaprone, polydioxanone, poly urethane, polyacrylonitrile, polyvinylacetate, copolymers of urethane/carbonate, copolymers of styrene/maleic acid, and mixtures or copolymer thereof.

According to one embodiment of the present invention, the present invention provides methods to apply the binder to the barbed suture. The binder can be dissolved in a solvent to form a solution. The solution described herein is prepared by conventional methods. It is usually prepared by, in a first step, adding sufficient amounts of binder to a solvent. Gentle heating and stirring can be used as necessary to promote dissolution of the binder into the solvent. Excessive heating should not be used in order to prevent degradation of the components. Then this solution is applied on the suture by coating, spraying, brushing or dipping to cover the barbs or bind the barbs to the suture thread. While the solution is applied on the suture, the solvent in the solution will evaporate rapidly to cover the barb or form a solid bond between barbs and suture thread. Alternatively, during this evaporation process, pressure may be applied to press the barbs against the main suture thread allowing the barbs to conform to the main suture thread. This can further reduce the cross sectional profile of the suture thread. The examples of the appropriate solvent are water, dimethylsulfoxide (DMSO), alcohols such as ethanol and aldehydes and ketones, such as acetone. Alternatively, the binder can be melted by heating above its melting temperature and then applied on the suture by coating, spraying, brushing or dipping. Then the binder cools down to cover the barb or form a solid bond between barbs and suture thread. In the other embodiment, it may be necessary to press the barbs against the main suture thread allowing the barb to conform to the main suture thread during the cooling. This can further reduce the cross sectional profile of the suture thread. Alternatively, some selected barbs on the barbed suture are bound to the suture thread by the binder. In another embodiment, rather than some selected barbs, all the barbs on the barbed suture are bound to the suture thread by the binder.

According to the other embodiment of the present invention, the present invention provides methods to apply the binder to the selected barbs on the barbed suture. In this invention, a plurality of the first set of barbs can be formed on a plurality of the first locations alone the length of the suture thread. The barbs can be formed either by attaching features on the suture thread or by cutting into the suture thread. Then the binder can be applied on the suture by spraying, brushing or dipping to bind the first set of barbs to the main suture thread after the binder is melted or dissolved in a solvent. After the binding between the first set of barbs and suture thread is completed, pluralities of the second set of barbs are formed on a plurality of the second locations along the length of the same barbed suture. The plurality of second locations is interspersed with the plurality of first locations along a length of said suture thread. The second set of barbs can also be formed either by attaching features on the suture thread or by cutting into the suture thread. Those second set of barbs are not restrained by the binder and are able to engage tissue during the insertion of the suture in the tissue providing instantaneous retaining strength to the suture. On the other hand, the bound first set of barbs will engage tissue with additional retaining strength after the binder is dissolved or degraded in the body. In one embodiment, the first set of barbs have barbs tips pointed to the insertion direction. Binding those barbs to the suture thread can reduce resistance against insertion. In the other embodiment, the first set of barbs are bidirectional barbs. Those barbs are bound to the suture thread by the binder regardless barb orientation to reduce resistance against insertion. In yet another embodiment, the second set of barbs have barbs tips pointed away from the insertion direction. Those un-bound barbs are able to engage tissue during the insertion of the suture in the tissue providing instantaneous retaining strength to the suture.

According to yet the other embodiment of the present invention, the present invention provides an alternative method to apply the binder to the selected barbs on the barbed suture. The barbs on the suture can be formed either by attaching features on the suture thread or by cutting into the suture thread. Then some of the barbs are covered or masked by a protective sheet or a protective coating to prevent those barbs from contacting with the binder. Only the un-masked barbs are exposed to the binding processes. After that, the binder is applied on the suture by spraying, brushing or dipping to bind the un-masked barbs to the main suture thread. When the binding process between the un-masked barbs and the suture thread is completed, the protective sheet or coating is removed from the suture to expose the masked barbs. Those masked and un-bound barbs are able to engage tissue during the insertion of the suture in the tissue providing instantaneous retaining strength to the suture. On the other hand, the un-masked and bound barbs on the same suture will engage tissue with additional retaining strength after the binder is dissolved or degraded in the body. In one embodiment, the un-masked barbs have barbs tips pointed to the insertion direction. Binding those barbs to the suture thread can reduce resistance against insertion. In the other embodiment, the un-masked barbs are bidirectional barbs. Those barbs are bound to the suture thread by the binder regardless barb orientation to reduce resistance against insertion. In yet another embodiment, the masked barbs have barbs tips pointed away from the insertion direction. Those un-bound barbs are able to engage tissue during the insertion of the suture in the tissue providing instantaneous retaining strength to the suture. In further another embodiment, the masked barbs are bidirectional barbs with barbs tips pointed to both directions of the suture thread.

According to further embodiment of the present invention, the present invention provides an alternative method to apply the binder to the barbs on the barbed suture. The barbs on the suture can be formed either by attaching features on the suture thread or by cutting into the suture thread. Immediately after each individual barb is formed, the binder is applied on that barb by coating, spraying, brushing or dipping to cover or bind the barb to the main suture thread. When the binding process between the barb and suture thread is completed, the barb is covered, restrained or conform to the suture thread. After the binding, the barbed suture is then sterilized with standard sterilization methods such as Gamma, E-Beam, ETO, or heat sterilization, etc. Alternatively, only selected barbs of the barbed suture are bound to the suture thread by the binder. In another embodiment, rather than some selectively-bound barbs, all the barbs of the barbed suture are bound to the suture thread by the binder. In yet another embodiment, only those barbs with barbs tips pointed toward the insertion direction are bound by the binder. Those barbs will engage tissue providing additional retaining strength to the suture after the binder is dissolved or degraded in the body. Those un-bound barbs are able to engage tissue during the insertion of the suture in the tissue providing instantaneous retaining strength to the suture.

Most of the barbed sutures disclosed here are described as having their ends being pointed and made of a material with sufficient stiffness to penetrate tissue. In yet the other embodiment of the present invention, the barbed suture is unidirectional and further comprises a delivery device that is linked to the insertion end of the barbed suture to assist the insertion of the suture in the tissue. The said delivery device may be a needle. In another embodiment of the present invention, the barbed suture is bidirectional and further comprises two delivery devices that are linked to both ends of the barbed suture. The said insertion devices are needles. The suture needle may be permanently attached or removably attached to the barbed surgical suture or, alternatively, the suture needle may be integrally formed with the barbed surgical suture.

In yet the other embodiment of the present invention, the present invention includes suture of a wide variety of materials and configurations. For example, the suture thread can be either monofilament or multiple filaments. The materials used for the suture can be either absorbable or non-absorbable. The barbed sutures can be unidirectional, or bidirectional with a range of various barb shapes, sizes designed for various tasks.

In one embodiment of this invention, this barbed suture is resorbable after the barbed suture is inserted in the body. Both the barb and the suture thread can be made by the same material. The resorbable barbed suture can be substantially degraded, excreted or metabolized by the body. This process may typically take up to several weeks and is regulated by variables in the composition and manufacturing of the barbed suture disclosed here. The degradation by-products may be mainly expelled out of the body via normal respiration and excretion. The materials useful for resorbable barbed suture include degradable polymers, metal, alloy, ceramic, etc. Examples of degradable polymers are polylactic acid polymer or copolymer, polyglycolic acid polymer or copolymer, copolymers of polylactic acid and polyglycolic acid, polyepsilon caprolactone polymer or copolymer, poly caprolactone polymer or copolymer, polyhydroxy butyric acid polymer or copolymer, poly-3-hydroxybutyrate polymer or copolymer, polyorthoesters polymer or copolymer, polyacetals polymer or copolymer, polydihydropyrans polymer or copolymer, poly trimethylene carbonate, collagen, hydrogel, hyaluronic acid, polyacrylamine, poly(vinylcarboxylic acid), polymethacrylic acid, polyacrylic acid polymer or copolymer, poly amino acids, fibrin, chitosan, gelatin, alginate, cellulose acetate phthalate, cellulosic, polylysine, polyarginine, poly aspartic acid, poly glutamic acid, methylcellulose, hydroxy ethyl cellulose, hydroxy propyl cellulose, hydroxypropyl methylcellulose, cellulose acetate, nitrocellulose, hypromellose, cellulose butyrate, polyhydroxyalkanoates, polyhydroxyvalerate (PHV) and polyhydroxyhexanoate, polyanhydrides, polyethylene terephthalate, polyhydroxurethanes, poly(2-hydroxyethyl-methacrylate), polyphosphazenes, polyphosphoesters, polyglactin, catgut, silk, poliglecaprone, polydioxanone, poly urethane, polyacrylonitrile, polyvinylacetate, copolymers of urethane/carbonate, copolymers of styrene/maleic acid, and mixtures or copolymer thereof. Alternatively, the barb and suture thread may be made by different materials.

In another embodiment of this invention, the barbed suture may be made from a non-resorbable material. The materials useful for non-resorbable barbed suture include non-biodegradable polymers, metal, alloy, silk, etc. Examples of non-biodegradable polymers are polypropylene, polyethylene, polyamide, polyester, polytetrafluoroethylene, polyether-ester, or polyurethane, etc. Alternatively, the barb and the suture thread may be made by different materials.

According to one embodiment of the present invention, the present invention provides methods to manufacture the barb. The barbs may be formed using any suitable method, including injection molding, extrusion, thermal forming, stamping, die cutting, laser cutting, etc. In regard to die cutting, filaments or ribbons are made by a typical molding process such as extrusion or injection molding. Then, the barbs are cut from the filaments or ribbons with a die and then bound on the surface of the suture thread. In another embodiment, the filaments can be made by extrusion, thermal forming, die cutting, machining or other forming techniques, etc. Then the filaments can be bent or shaped into barbs before they are bound on the surface of the suture thread. Alternatively, the formation and bending of the filaments into barbs can be done in one step.

The binding of the barb to the suture thread can be done by traditional methods such as contact welding, ultrasonic welding, hot air welding, hot plate welding, extrusion welding, induction welding, speed tip welding, RF welding, friction welding, laser welding, adhesive, etc. For an example of some embodiments in this invention, the contact welding is usually prepared by, in a first step, increasing the contact surface of the end of the barb by shaping it into, for example, a rectangular cross section. Other shapes can also be used for the contact surface, such as square, circular, or oblong. Large contact surface can enhance the bonding strength between the barb and suture thread. This can be done by placing the end in a hot press and compressing the end with the help of heat. Alternatively, the rectangular cross section of the end can be formed during the barb fabrication step when the barb is cut from a thin sheet. In a second step, the end of the barb is bent to form an acute angle from the barb axis. As a consequence, the contact surface between the barb end and the suture thread can be increased further when the barb end is coaxial with the suture thread, and the barb tip is in an acute angle with the suture thread as shown in FIG. 1.

Alternatively, the bending of the end can be done during the barb fabrication step when the barb is cut from a thin sheet. In a third step, approximating and aligning the end of the barb to the thread surface. In a fourth step, a heated welding rod is applied on the end of the barb to melt and fuse the end to the suture thread. Gentle heating is necessary to effect the binding without adversely change the structure of the suture thread. Excessive heating may also cause degradation of the suture thread and barb. In a fifth step, the barb suture is cooled to allow solidification of the bond. After the binding, the barbed suture is then sterilized with standard sterilization methods such as Gamma, E-Beam, ETO, or heat sterilization, etc.

According to some embodiments of the present invention, the barbed suture of the present invention comprises a contrast agent which provides visibility of the binder via fluoroscopy or ultrasound. The radiopaque or ultrasound contrast agent is either water soluble or insoluble. Examples of water insoluble radiopaque contrast agents include tantalum, tungsten, gold, platinum and barium compounds. Examples of water soluble radiopaque contrast agents include metrizamide, iopamidol, jothalamate sodium, jodomide sodium, and meglumine. Examples of ultrasound contrast agents include microbubbles, air bubbles, gas bubbles, CO2, O2, N2 and air.

According to some embodiments of the present invention, the barbed suture of the present invention comprises a medicament including, for example, medicament to promote healing, treat diseases, prevent infection, reduce pain, reduce scar, etc. Example of such medicament may include angiogenesis inhibiting compounds, steroidal or non-steroidal anti-inflammatory agents, thrombotic agents, growth factors, anti-proliferative agents, anti-infective agents, fibrosis-inducing agents, anti-scarring agents, analgesics, and anti-microtubule agents, etc. Those medicaments can be applied in the barbed suture by many methods. They can be dissolved in a solvent and then mixed with a binder, or they can be dissolved directly in a solvent without binder. Then the medicament can be applied to the barbed suture by spraying, dipping, coating, brushing, etc. After the barbed suture is inserted in the body, the medicament will be dissolved by body fluid releasing the medicament in the body to treat the wound or illness. The purpose of the barbed suture may also determine the sort of medicament that is applied on the barbed suture. For example, the barbed suture with anti-inflammatory agent is suitable for wound closure. The barbed suture with angiogenesis inhibiting compound is suitable for tumor excision surgery. In one embodiment of this invention, more than one medicament can be used in the barbed suture. In the other embodiment of this invention, more than one medicament can be used in various section of the barbed suture. For example, one section of the barbed suture comprises anti-inflammatory agent, and the other section of the barbed suture may comprise anti-scarring agent.

In summary, according to the embodiments of the present invention, the present invention provides a new barbed suture with improved retaining strength within the tissue in both directions of the barbed suture. The barbed suture of the present invention comprises a barb which is flexible and is able to conform to the main body of the suture thread to prevent the barb from penetrating the tissue. As a result, the barbed suture is allowed to be readily deployed in the tissue without damaging the surrounding tissue. After the suture is placed in the tissue, the tissue ingrowth in the barbed suture increased retention strength against suture movement in both directions of the suture. This can enhance the barbed suture's ability to hold tissue together with improved wound closure or tissue approximation performance.

It is understood that this disclosure, in many respects, is only illustrative of the numerous alternative device embodiments of the present invention. Changes may be made in the details, particularly in matters of shape, size, material and arrangement of various device components without exceeding the scope of the various embodiments of the invention. Those skilled in the art will appreciate that the exemplary embodiments and descriptions thereof are merely illustrative of the invention as a whole. While several principles of the invention are made clear in the exemplary embodiments described above, those skilled in the art will appreciate that modifications of the structure, arrangement, proportions, elements, materials and methods of use, may be utilized in the practice of the invention, and otherwise, which are particularly adapted to specific environments and operative requirements without departing from the scope of the invention. In addition, while certain features and elements have been described in connection with particular embodiments, those skilled in the art will appreciate that those features and elements can be combined with the other embodiments disclosed herein

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A barbed suture, comprising:
a suture thread with a central axis; and
a plurality of barbs disposed on a surface of said suture thread;
wherein each barb comprises a substantially V-shaped filament with a peak and two ends, said two ends of said filament are attached to said suture thread and spaced apart from each other, said peak of said filament is configured to penetrate tissue;
wherein each barb is oriented in an acute angle to said central axis of said suture thread.

2. The barbed suture according to claim 1, wherein successive barbs of said plurality of barbs are circumferentially disposed around said suture thread.

3. The barbed suture according to claim 1, wherein successive barbs of said plurality of barbs are axially spaced apart along the central axis of said suture thread.

4. The barbed suture according to claim 1, wherein successive barbs of said plurality of barbs are circumferentially disposed around said suture thread and axially spaced apart along the central axis of said suture thread.

5. The barbed suture according to claim 1, wherein successive barbs of said plurality of barbs are disposed spirally on the surface of said suture thread.

6. The barbed suture according to claim 1, wherein the ends of each barb are connected to the ends of adjacent barbs.

7. The barbed suture according to claim 1, wherein at least one barb overlaps with an adjacent barb on the surface of said suture thread.

8. The barbed suture according to claim 1, wherein the plurality of barbs are interconnected.

9. The barbed suture according to claim 1, wherein said plurality of barbs overlap and form a lattice structure.

10. The barbed suture according to claim 1, wherein each barb forms an opening that is configured to allow tissue ingrowth.

11. The barbed suture according to claim 1, wherein said two ends of said filament are attached to said suture thread by a method selected from contact welding, ultrasonic welding, hot air welding, induction welding, speed tip welding, RF welding, friction welding, laser welding, and adhesive.

12. The barbed suture according to claim 1, wherein successive barbs of said plurality of barbs are separated by a predetermined distance along a length of said suture thread, the predetermined distance between approximately 0.01 to 1.0 cm.

13. The barbed suture according to claim 1, wherein the length of each barb is between approximately 0.01 to 1.0 cm.

14. The barbed suture according to claim 1, wherein the thickness of each barb is between approximately 0.01 to 0.8 cm.

15. The barbed suture according to claim 1, wherein the ends of the V-shaped filament are spaced apart at a distance approximately ¼, ⅓, ½, ⅔ or 3/3 of the width of the suture thread.

16. The barbed suture according to claim 1, wherein said suture thread and/or said plurality of barbs comprises material selected from the group consisting of degradable polymer, non-degradable polymer, metal, metal alloy, ceramic, silk, and mixtures thereof.

17. The barbed suture according to claim 1, further comprising a binder, wherein said binder temporarily binds said plurality of barbs to said suture thread into a delivery configuration where the plurality of barbs are pressed against the surface of the suture thread, wherein said binder is soluble or degradable in a patient's body and configured to release said plurality of barbs into a deployed configuration where the plurality of barbs are configured to extend radially outwards in order to resist movement of said suture thread in the patient's body.

18. A barbed suture, comprising:
a suture thread having a central axis; and
a filament having a plurality of peaks and valleys, the filament disposed on a surface of said suture thread;
wherein said valleys of said filament are attached to said suture thread;
wherein said peaks of said filament form barbs that are oriented in an acute angle to said central axis of said suture thread, said barbs configured to penetrate tissue.

19. The barbed suture according to claim 18, further comprising a binder, wherein said binder temporarily binds said filament to said suture thread into a delivery configuration where the barbs of the filament are pressed against the surface of the suture thread, wherein said binder is soluble or degradable in a patient's body and configured to release said barbs into a deployed configuration where the barbs are configured to extend radially outwards in order to resist movement of said suture thread in the patient's body.

20. A method to make a barbed suture, comprising:
forming a suture thread having a central axis and a surface;
forming a substantially V-shaped filament having a tissue penetration peak and two ends; and
attaching said two ends of said filament to said surface of said suture thread such that said two ends are spaced apart from each other by a predetermined distance and said peak is oriented at an acute angle to the central axis of said suture thread.
